(19) United States
(12) Patent Application Publication
(10) Pub. No.: US 2025/0312080 A1
(43) Pub. Date: Oct. 9, 2025

(54) SYSTEM TO CREATE CELLULAR LYSIS AND DELIVER FLUIDS INTRATUMORALLY

(71) Applicant: Syncromune, Inc., Fort Lauderdale, FL (US)

(72) Inventors: Daniel K. Recinella, Waynesville, NC (US); Eamonn P. Hobbs, Fort Lauderdale, FL (US); Stephen T. Kee, Laghey (IE); Timothy B. Gonzalez, Coral Springs, FL (US)

(21) Appl. No.: 19/171,119

(22) Filed: Apr. 4, 2025

Related U.S. Application Data
(60) Provisional application No. 63/575,527, filed on Apr. 5, 2024.

(51) Int. Cl. A61B 18/02; A61B 18/00

(52) U.S. Cl. CPC A61B 18/02; A61B 2018/00577; A61B 2018/0293

(57) ABSTRACT

The present disclosure provides a system that incorporates a design that has an energy source and probe for creating cellular lysis and in integral infusion sheath for directing infusion of fluids into the same target where the cellular lysis was performed. The system includes an energy probe and an infusion sheath. The combined system provides accurate drug infusion into an area of cellular lysis while removing physical manipulation required for other systems where the cellular lysis energy source and the infusion sheath are separate.

### CROSS-REFERENCE TO RELATED APPLICATIONS

This application claims the benefit of, and priority to, U.S. Provisional Application No. 63/575,527, filed Apr. 5, 2024, the entire contents of which are incorporated herein by reference in its entirety.

### FIELD OF INVENTION

Systems, methods, and devices of this disclosure relate to cellular lysis treatment and targeted drug infusion post treatment in the field of cancer immunotherapy.

### BACKGROUND

Current clinical approaches for cancer immunotherapy rely on the systemic administration of high doses of one or two biological immunomodulatory agents. Major advances in the treatment of cancer have been made in the last decade by the use of monoclonal antibodies (mAbs) that block immune checkpoint inhibitor molecules such as PD-1, PD-L1 and CTLA-4. However, despite the significant benefits obtained by the use of these mAbs, a substantial fraction of patients still do not respond to the therapy or become refractory to it.

Additionally, the use of high systemic doses of these checkpoint inhibitors may be accompanied by high frequency of adverse events, particularly when used in combination with other immunotherapeutic agents. This may limit the number of agents and doses that can be combined to address the multifactorial nature of tumor immune resistance.

### BRIEF SUMMARY

Systems, methods, and devices described herein relate to, a system for collocating an energy for cell lysis and an infusion of a fluid in a target site including tissue of a subject, the system includes: an energy probe configured to emit an energy into the tissue of the target site thereby causing cell lysis of said tissue, and an infusion sheath having a lumen configured to at least partially surround the energy probe, where the infusion sheath is configured to provide a pathway for the fluid to travel distally within the lumen and exit a distal portion of the infusion sheath, where the probe is coaxial with the infusion sheath and shares a longitudinal axis thereby allowing co-location of the energy from the probe and the infusion of the fluid from the infusion sheath in the target site. In some embodiments, the infusion sheath includes at least one port in the distal portion of the infusion sheath that is in fluid communication with the pathway of the infusion sheath. In some embodiments, the at least one port is a slit. In some embodiments, the energy probe and the infusion sheath are translatable relative to one another along the longitudinal axis. In some embodiments, the pathway and port are configured to deliver the fluid while the probe is within the pathway at the distal end of the infusion sheath. In some embodiments, the system includes a valve configured to eliminate proximal flow of the fluid during and/or following delivery thereof to the target site. In some embodiments, the system includes a tube in fluid communication with the pathway of the infusion sheath at a distal end of the tube and in fluid communication with a source of the fluid at a proximal end of the tube. In some embodiments, the fluid is a therapeutic agent effective in treating the tissue at the tissue site. In some embodiments, the energy induces cryolysis of tissue at the target site. In some embodiments, the system includes a location indicator configured to provide a visual, tactile, or audible indication of the probe's position relative to the infusion sheath's position or vice versa. In some embodiments, the system includes a location indicator configured to provide a visual, tactile, or audible indication of a position of a port of the infusion sheath relative to the probe's position, or vice versa. In some embodiments, the system includes a channel disposed in the exterior surface of the energy probe, the channel configured to slidably couple with a protrusion extending from the interior wall of the infusion sheath, thereby preventing circumferential rotation of the infusion sheath relative to the energy probe. In some embodiments, the system includes a channel disposed at least partially through a wall of the infusion sheath, the channel configured to slidably couple with a protrusion extending from the exterior surface of the energy probe, thereby preventing circumferential rotation of the infusion sheath relative to the energy probe. In some embodiments, the system includes a valve configured to connect an infusion tube with a proximal end of the infusion sheath, so as to provide the infusion of the fluid to the enter lumen of the infusion sheath and/or a plurality of fluid channels disposed within the walls of the sheath. In some embodiments, the infusion sheath has a first inner diameter located at a proximal end of the infusion sheath and a second inner diameter located at a distal end of the infusion sheath. In some embodiments, the system includes an indicator disposed at least partially on or about the energy probe. In some embodiments, the indicator is an audio indicator configured to notify a user that the distal end of the infusion sheath has been advanced to a location relative to the probe that co-locates the fluid delivery with the energy delivered by the probe. In some embodiments, the indicator marks a target location on a proximal end of the system to slidably position the infusion sheath relative to the energy probe to deliver the fluid in a location that is co-located with the energy delivery location of the probe. In some embodiments, the infusion sheath is slidable relative to the longitudinal axis of the energy probe. In some embodiments, the energy probe is slidable relative to the longitudinal axis of the infusion sheath. In some embodiments, the energy probe is a cryoprobe. In some embodiments, the infusion sheath includes a plurality of fluid pathways disposed within a wall of the infusion sheath, where the fluid pathways are configured to release the infusion of the therapeutic agent to flow from a first location of the infusion sheath through a second location of the infusion sheath spaced apart from and located distal to the first location. In some embodiments, the second location is flush with the distal end of the infusion sheath. In some embodiments, the at least one port is an annular orifice. In some embodiments, the infusion sheath includes a polymer. In some embodiments, the infusion sheath includes a metal. In some embodiments, the distance the infusion sheath is slidably moved over the energy probe or the distance the energy probe is moved while at least partially within the infusion sheath is controlled by moving a guide within a slot to a target distance, where the guide extends from the energy probe or the infusion sheath and the slot is at least partially disposed in or extending from the corresponding surface of the energy probe or the infusion sheath. In some embodiments, the infusion sheath has a first inner diameter located at a proximal end of the infusion sheath and a second inner diameter located at a distal end of the infusion sheath. In some embodiments, the infusion sheath includes a plurality of fluid pathways disposed through a wall of the infusion sheath, where the fluid pathways are in fluid communication with a source of the fluid at the proximal end of the system, and each pathway is in fluid communication with an associated opening at the distal end of the infusion sheath. Other technical features may be readily apparent to one skilled in the art from the following figures, descriptions, and claims.

Systems, methods, and devices described herein relate to, a method for co-locating energy delivery and fluid delivery to tissue at a target site in a patient, the method includes: disposing a cryolysis system includes an energy probe coaxial with and positioned within a lumen of an infusion sheath at least partially into a patient such that a distal end of the energy probe is disposed in the target site, providing energy from the energy probe to the tissue thereby lysing at least a portion of a plurality of cells in the target site, slidably moving the infusion sheath in a distal direction along a longitudinal axis of the energy probe from a first position to a second position, where the second position is spaced apart from the first position, delivering an infusion of a fluid to the target site through the infusion sheath, and removing the infusion sheath and the energy probe from the target site. The method in some embodiments, includes delivering the fluid out one or more openings in the distal end of the infusion sheath, where such openings are disposed radially through a wall of the infusion sheath to be in fluid communication with a fluid pathway within a lumen of the infusion sheath that extends to a proximal end of the cryolysis system to a fluid source. The method in some embodiments, where delivering the fluid and slidably moving the infusion sheath are done concurrently. The method in some embodiments, where the infusion sheath is in the third position when disposed at least partially into the patient. The method in some embodiments, where a distal end of the infusion sheath is located distal to a distal end of the energy probe when in the third position. The method in some embodiments, where a distal end of the infusion sheath is located proximal to a distal end of the energy probe when in the third position. The method in some embodiments, where a distal end of the infusion sheath is located at a location distal to the distal end of the energy probe when in the second position. The method in some embodiments, where a distal end of the infusion sheath is located at a location proximal to the distal end of the energy probe when in the second position. The method in some embodiments, includes sliding a protrusion extending from the exterior wall of the energy probe within a channel disposed in the infusion sheath to slidably move the infusion sheath from the first position to the second position, where the channel is configured to slidably couple with the protrusion, and optionally, where the protrusion coupled with the channel is configured to prevent circumferential rotation of the infusion sheath relative to the energy probe. The method in some embodiments, includes sliding a protrusion extending from the exterior surface of the energy probe within a channel disposed at least partially through a wall of the infusion sheath to slidably move the infusion sheath from the first position to the second position, where the channel is configured to slidably couple with the protrusion, and optionally, where the protrusion coupled with the channel is configured to prevent circumferential rotation of the infusion sheath relative to the energy probe. The method in some embodiments, includes determining the second position by aligning a portion of the proximal end of the sheath relative to a portion of a proximal end of probe according to an audio, tactile, or visual indicator. The method in some embodiments, where delivering the infusion of a therapeutic agent to the target tissue occurs through a valve configured to connect an infusion tube with a proximal end of the infusion sheath, so as to provide fluid through a pathway within the infusion sheath and out the one or more openings of the infusion sheath. The method in some embodiments, where the energy probe is a cryoprobe and providing energy to the tissue at the tissue site at least partially freezes the tissue thereby lysing at least a portion of the plurality of cells in the target site. The method in some embodiments, where delivering the infusion of a fluid to the target tissue includes opening a valve at the proximal end of the system in the fluid pathway of the fluid that connects an infusion fluid source with a proximal end of the infusion sheath. The method in some embodiments, where the infusion sheath is configured to form a seal with the energy probe when in the first position such that the infusion of therapeutic agent is retained in the infusion sheath, and where the infusion sheath is configured to deliver the infusion of therapeutic agent to the target tissue when in the second position. The method in some embodiments, where a distal end of the infusion sheath is located proximal to the distal end of the energy probe when in the first position. Other technical features may be readily apparent to one skilled in the art from the following figures, descriptions, and claims. In some embodiments, the therapeutic agent is a cancer therapeutic agent. In some embodiments, the first inner diameter is larger than the second inner diameter, and where the first inner diameter tapers to the second inner diameter in a location between a proximal end of the infusion sheath and the distal end of the infusion sheath. In some embodiments, the second inner diameter forms a fluid tight seal with the outer diameter of the energy probe, such that when a portion of the energy probe is in a first position disposed within the first region of the infusion sheath having the second inner diameter, the fluid does not flow from the inner lumen of the infusion sheath through the distal end of the infusion sheath, and where, when the energy probe is in a second position at least partially disposed the second region of the infusion sheath having the first inner diameter and not in the first region of the infusion sheath the infusion sheath having the second inner diameter, the fluid in the inner lumen of the infusion sheath is in fluid communication with the target site. In some embodiments, the indicator is a visual distance and/or rotational indicator located on the system that indicates to a user that a distal end of the infusion sheath has been advanced to a location relative to the probe that co-locates the fluid delivery with the energy delivered by the probe. In some embodiments, the first inner diameter is larger than the second inner diameter, and where the first inner diameter tapers to the second inner diameter in a location between the proximal end of the infusion sheath and the distal end of the infusion sheath. In some embodiments, the second inner diameter is substantially the same as the outer diameter of the energy probe, such that when a portion of the energy probe is disposed within the second inner diameter, fluid does not flow from the inner lumen of the infusion sheath through the distal end of the infusion sheath, and where the first inner diameter is larger than the outer diameter of the energy probe, such that when the energy probe is at least partially disposed only in the portion of the energy probe having the first inner diameter, the inner lumen of the infusion sheath is in fluid communication with the target site. In some embodiments, the opening is selected from a group consisting of a slit and an annular orifice. The method in some embodiments, where the one or more openings is a slit. The method in some embodiments, includes slidably moving the infusion sheath in a proximal direction along the longitudinal axis of the energy probe from the second toward the first position, where delivering the fluid and slidably moving in this proximal direction are done concurrently. The method in some embodiments, includes slidably moving the infusion sheath distal to the second position to a third position, where when in such third position buckling of the infusion sheath is reduced relative to when in the first or second positions and/or mechanical strength of the system at the location of the energy probe is enhanced relative to the energy probe alone without the sheath distal advancement, where the enhanced mechanical strength is provided about the distal end of the energy probe upon disposing the cryolysis system in the patient. The method in some embodiments, where the indicator marks a target location to slidably align the infusion sheath and the energy probe thereby co-locating delivery of the fluid with the energy delivered by the probe. Other technical features may be readily apparent to one skilled in the art from the following figures, descriptions, and claims.

### BRIEF DESCRIPTION OF THE SEVERAL VIEWS OF THE DRAWINGS

Figures illustrated herein are exemplary block drawings showing relative positions of certain elements described herein. However, the figures do not show the exact couplings or connections that may or may not exist between elements. Additionally, in some drawings, internal and external features may be shown in the same renderings for conceptualizing the overall concepts and not for conceptualizing them from an engineering perspective.

FIG. 1A illustrates an example of a cryolysis system in a first position comprising a probe and an infusion sheath comprising at least one port, according to embodiments described herein.

FIG. 1B illustrates an example of the cryolysis system shown in FIG. 1A in a second position, according to embodiments described herein.

FIG. 2A illustrates an example of a cryolysis system in a first position comprising a probe and an infusion sheath having a plurality of lumens disposed in the wall of the infusion sheath, according to embodiments described herein.

FIG. 2B illustrates an example of the cryolysis system shown in FIG. 2A in a second position, according to embodiments described herein.

FIG. 3A illustrates an example of a cryolysis system in a first position comprising a probe and an infusion sheath having two or more inner diameters and a slide coupled with a channel, according to embodiments described herein.

FIG. 3B illustrates an example of a cutaway view of the cryolysis system shown in FIG. 3A with the energy probe extended beyond the infusion sheath, according to embodiments described herein.

FIG. 3C illustrates an example of a second cutaway view of the cryolysis system shown in FIG. 3A in a second position, according to embodiments described herein.

FIG. 4A illustrates an example of an energy probe and an infusion sheath system inserted into a target tissue, according to embodiments described herein.

FIG. 4B illustrates an example of the energy probe and infusion sheath of FIG. 4A delivering energy to the target tissue site, according to embodiments described herein.

FIG. 4C illustrates an example of the area of cellular lysis created from the energy that was delivered by the energy probe and infusion sheath of FIG. 4B in the target zone of the target tissue, according to embodiments described herein.

FIG. 4D illustrates an example of the energy probe and infusion sheath of FIG. 4C with the infusion sheath positioned in the same target location that has experienced cellular lysis, and delivering therapeutic agent in the same area according to embodiments described herein.

FIG. 5A illustrates an example of a cryolysis system comprising an energy probe and an infusion sheath in a first position, according to embodiments described herein.

FIG. 5B illustrates a second view of the cryolysis system shown in FIG. 5A in a second position, according to embodiments described herein.

### DETAILED DESCRIPTION

As used herein, the term “about” means within ±10% of the value it modifies. For example, “about 1” means “0.9 to 1.1”, “about 2%” means “1.8% to 2.2%”, “about 2% to 3%” means “1.8% to 3.3%”, and “about 3% to about 4%” means “2.7% to 4.4%.” Unless otherwise clear from the context, all numerical values provided herein are modified by the term “about”.

As used herein, the term “cellular lysis” refers to the breaking down of the membrane of a cell, through the application of energy to a targeted area, and encompasses diverse mechanisms of cell death including necrosis, necroptosis, apoptosis, pyroptosis, etc. For example, this action causes the contents of the cells, to be released into the micro-environment surrounding the lysed area and initiate an immune response by diverse mechanisms.

As used herein, the term “cryolysis” refers to a process that uses localized, extreme cold to partially destroy a target tissue, e.g., an abnormal or diseased tissue, such as a tumor. Thus, cryolysis, as performed in methods described herein, is similar to cryoablation, but cryolysis does not aim to ablate an entire tumor. Accordingly, during administration of a therapy that causes cell lysis by cryolysis, cryoprobes are positioned adjacent to, or within (e.g., intratumoral positioning), a target tissue in such a way that a freezing process will partially destroy abnormal or diseased tissue, such as a tumor. In some embodiments of the present disclosure, a goal of cryolysis is not to achieve full ablation of the tumor tissue (i.e., cryoablation), but to elicit a partial necrotic zone within a tumor to release sufficient amounts of tumor specific antigens (TSA), tumor associated antigens (TAA) and damage associated molecular patterns (DAMPs) to initiate an immune response. Accordingly, cryolysis may differ from cryoablation in methods of administration including, but not limited to, minimum temperature achieved in a target tissue treatment zone, rate of cooling of a target tissue treatment zone (which is related to, among other things, rate of cooling of a cryotherapy device, such as a cryoprobe), diameter of an ice ball formed at a target tissue treatment zone, diameter of an isotherm within an ice ball that reaches a critical temperature for necrosis and apoptosis, duty cycle of a cryotherapy device, speed of thaw, and duration of freeze-thaw cycles

As used herein, the term “cryoprobe” refers to a hollow needle or elongated tube through which a cooled, thermally conductive, fluid is circulated. A thermally conductive fluid may be any acceptable cryogen used in the field, for example, argon, nitrous oxide, carbon dioxide, or liquid nitrogen.

As used herein, the term “energy probe” or “probe” as used herein refers to any structure that is designed to be inserted into soft tissue or bone and is used to transmit energy from an energy source to a target site. For example, the energy probe may facilitate cellular lysis by applying the energy to the surrounding tissue. In some embodiments, this energy source is a cryoprobe. In some embodiments, the energy probe is for creating Irreversible Electroporation (IRE) in the target tissue. In some embodiments the energy probe is for delivering Pulsed Electric Fields (PEF) to the target tissue. A cryoprobe is an example of an energy probe. The terms “probe,” and “energy probe” are used interchangeably throughout this disclosure. As used herein the term “probe” is an energy probe. Alternatives and embodiments of energy probes are described further infra.

As used herein, the term “energy source” refers to any system that generates a form of energy that can be transmitted. For example, the energy source may be selected from a group consisting of radiofrequency, microwave, ultrasound, cryo, electrical, any other energy source known to those skilled in the art, and any combination thereof applied to a structure, such as a probe.

As used herein, the term “infusion sheath” or, synonymously, “infusible sheath,” refers to a channel where the flow of fluid is established. For example, this sheath may comprise different materials such as polymer or metal and can be of varying design such as a single tube, or a multi-lumen tube. A characteristic that defines the infusion sheath is the ability for fluid to flow through an inlet that is then carried distally through an inner portion of the sheath having an egress to thereby permit the flow of fluid out of the sheath and into the target site. Alternatives and embodiments of infusion sheaths are described further infra.

As used herein the terms “drug”, biologic and “therapeutic agent” may be used interchangeably and refer to pharmacologically active molecules that are used to treat, or prevent diseases or pathological conditions in a physiological system (e.g., a subject, or in vivo, in vitro, or ex vivo cells, tissues, and organs). Drugs act by altering the physiology of a living organism, tissue, cell, or in vitro system to which the drug has been administered. It is intended that the terms “drug” and “chemotherapeutic agent” encompass anti-hyperproliferative and antineoplastic compounds as well as other biologically therapeutic compounds.

As used herein, the term “oncolysis” refers to a modified state of tissue within a tumor where a portion of cells have been lysed and their contents have flowed into the surrounding environment. This occurs while, at the same time, portions of the target area remain intact with pathways, such as lymphatics, still being viable for transporting material from the tumor environment into the lymphatic system.

As used herein, the terms “subject” or “patient” may be used interchangeably and refer to any animal, including a mammal, to which a provided method and/or composition is or may be administered, e.g., for experimental, diagnostic, prophylactic, and/or therapeutic purposes. The term “mammal” as used herein, encompasses any mammal. Examples of mammals include, but are not limited to, cows, horses, sheep, pigs, cats, dogs, mice, rats, rabbits, guinea pigs, monkeys, humans, etc. The subject is preferably a human. In some embodiments a subject is suffering from or is susceptible to one or more disorders or conditions. In some embodiments, a subject displays one or more symptoms of a disorder or condition. In some embodiments, a subject has been diagnosed with one or more disorders or conditions. In some embodiments, a disorder or condition is or includes a proliferative disease such as cancer. In some embodiments, a cancer is a solid tumor cancer. In some embodiments, a subject is receiving or has received certain therapy to diagnose and/or to treat a disease, disorder, or condition.

Traditional cancer immunotherapeutic approaches may provide benefits to a limited fraction of patients. For example, this may be due to systemic delivery of drugs which is limited in effectiveness and is associated with toxicity and adverse events. However, by delivering drugs intratumorally the amount of drug required is reduced compared to systemic delivery. Intratumoral delivery immediately following cellular lysis may carry the intracellular components, i.e., tumor specific antigens (TSA), tumor associated antigens (TAA), damage associated molecular patterns (DAMPs) and other cellular components released by the breakdown of the cellular wall, into the tumor environment and lymphatic system thereby initiating an immune response.

Another approach includes applying energy to the tumor locally. In some embodiments, different energy sources and probes are used to create cellular lysis. In some embodiments, the energy source used to create cellular lysis is a cryoprobe. Cryolysis, as described herein, may refer to a process that uses localized, extreme cold to partially destroy a target tissue, e.g., an abnormal or diseased tissue, such as a tumor. Thus, cryolysis, as performed in methods described herein, may be similar to cryoablation, but cryolysis does not aim to ablate an entire tumor. For example, a goal of cryolysis may not be to achieve full ablation of the tumor tissue (i.e., cryoablation), but to elicit a partial necrotic zone within a tumor to release sufficient amounts of tumor specific antigens (TSA), tumor associated antigens (TAA) and damage associated molecular patterns (DAMPs) to initiate an immune response. In some embodiments, fluid pathways within the tumor environment are maintained and not destroyed as would be the case with cryoablation.

However, traditional approaches of localized treatment of cancer by injecting drugs locally have not been optimized, because they fail to infuse drug in the same location where the energy source was placed. Surprisingly, as described herein, by infusing drug into a cancer target site (i.e., intratumoral administration) immediately post cell lysis with energy and accurately in the center of the treatment zone of the target site may result in optimization of the entrainment of the released antigens and other cellular components. Additionally, intratumoral administration may allow for a significant reduction in dose, which reduces cost and the associated risk of toxicity and occurrence of an adverse event. In some embodiments, the reduced dose of drug is delivered accurately into the target cellular lysis zone in order to effectively “wash” the tumor micro-environment of cellular components that has flowed into the area due to the cellular lysis treatment.

Systems, devices, and methods as described herein relate to the infusion of an immunotherapeutic drug into the same target site location of the cryolysis treatment. In some embodiments, the entrainment of cellular components into the peritumor lymphatic system is optimized via infusion of immunotherapeutic drug into the same target site location of the cryolysis treatment.

A principle of hydrodynamics is that liquids will flow from areas of higher pressure to areas of lower pressure, via the paths of least resistance and highest compliance. In some embodiments, during an infusion the target tissues exhibit resistance and compliance along a varying scale of elasticity. In some embodiments, the paths of least resistance change as the various pathways change in resistance and compliance. In some embodiments, the paths of least resistance become saturated with fluid and exhibit increasing resistance.

Normal, healthy tissue primarily uses the lymphatic drainage system to maintain fluid balance within the tissue. The lymphatic drainage vessels are typically the primary drainage pathway for interstitial and extravasated fluid. When the lymphatic drainage is compromised, edema may occur as a result. Metastatic tumors may extensively utilize the lymphatic system to traffic and message. For example, metastatic tumors are often hyperlymphatic when compared to healthy tissue or to earlier stage tumors. Tumors may be associated with tertiary lymphoid structures (TLS) either intratumorally, peritumorally, or in nearby tissue. TLS may communicate with the tumor and with secondary lymphoid structures such as tumor draining lymph nodes (TDLNs) via lymphatic vessels. The lymphatic tissue drainage system may present a significant path of least resistance for the tumor tissue resolving the interstitial, extravasated fluid overload that is created by the drug infusion into the treatment zone. Fluid flow through tissue medium has been modeled with Darcy's Law. In the context of cancer, Darcy's Law is linked to the interstitial fluid, the interstitial fluid pressure, and the hydraulic conductivity of the tumor environment, despite being originally derived to calculate flow through porous media. Darcy's Law describes that fluid flow will occur from a high-pressure area to a low-pressure area, based on the pressure difference and the hydraulic conductance of the medium. Hydraulic conductance is the capacity of a porous material to allow fluid to flow under the effect of pressure differentials. Higher pressure differentials between fluid inlets and outlets may create higher fluid flowrates.

Intratumoral vasculature may be tortuous, poorly organized, and have inconsistent vessel diameters with more prevalent branching compared to other normal tissue. For example, the vasculature and lymphatics within the center of the tumor are compressed due to the interstitial pressure within the tumor. The compression may create a high-pressure localization within the center of the tumor, thereby driving fluid flow within the tumor to the low-pressure draining system, represented by the peritumoral lymphatic vessels. Following Darcy's Law, the path from the center of the tumor to the peritumoral lymphatic vessels is the path of least resistance for fluid to flow from the tumor.

In some embodiments, an infusion rate between 1-3 ml/min of immunotherapeutic drug to the target site is used. The infusion rate may be selected to optimize for immunotherapeutic drug to first mix with the relatively lower resistance immediately surrounding lysed cell debris in the treatment zone. In some embodiments, the lysed cell debris includes tumor antigens, DAMPS, and other cellular contents. In some embodiments, once the lysed treatment zone has been saturated, the infused immunotherapeutic drug will flow via the paths of least resistance into the surrounding intact tumor tissue. In some embodiments, the infused immunotherapeutic drug also exits via the first available lymphatic vessel drainage pathway and blood vessel pathways. When the compliance limit of the tissue is reached, the continued infusion pressure reaches an equilibrium, where outgoing flow volume out of the tumor via the draining lymphatic vessels and blood vessels is approximately equal to the incoming flow volume being infused into the tumor. In some embodiments, the equilibrium point varies based on the intratumoral and peritumoral tissue compliance. In some embodiments, the equilibrium point will be reached at or before 5 ml of drug has been infused.

In some embodiments, at the equilibrium point, the continued infusion flow will saturate and distend the tumor tissue to a limit of its compliance. In some embodiments, the infused drug and any diffusible entities have mixed with and washed out into the surrounding intratumoral tissue, peritumoral tissue, and/or lymphatic drainage vessels. In some embodiments, the diffusible entities include lysed tumor cell contents, tumor antigens, DAMPS, and any combination thereof. In some embodiments, most of the excess fluid mixture of infused drug and diffusible entities drain out of the treatment zone and reach their cellular targets within the tumor microenvironment, TLSs, and TDLNs via the draining lymphatic vessels. In some embodiments, when the infusion is completed, the pressure head of the infusion is terminated and the distended and elastic intratumoral and peritumoral tissues drain the mixture of excess drug and debris fluid via the path of least resistance and return to their resting, pre-injection pressures. In some embodiments, the path of least resistance is the lymphatic vessel drainage system.

Current systems designed for intratumoral infusion of immunotherapeutic drugs alone include a multi-side hole needle with a closed distal end that creates jets of infusate. However, these systems are separate of the energy probe and placed independently. With this type of system, locating the end of the needle in the same location where the energy system was deployed may not be accurate and may allow for errors in placement, making the infusion of liquid miss the intended target.

Another current method of injecting immunostimulatory agents intratumorally includes placing a needle into the tumor and injecting without delivering energy prior to injections. With this method, accuracy of placement for infusion is not considered and the resulting drug infusion is inefficient.

A method of creating lysis by using temperatures around-40° C. and then delivering an effective amount of selected antigen presenting cells intratumorally or in proximity to the tumor or cancerous tissue when the bioavailability of the cancer-specific antigens in the bloodstream is at the maximum value has also been explored. In this method, the infusion may not be done within the same target area where the energy is delivered and not performed immediately post energy delivery. Therefore, the accuracy of infusion may not be considered and may have a deleterious effect on the outcome of the treatment.

Current methods of cellular ablation such as irreversible electroporation (IRE) and pulsed electric fields (PEF) aim to cause cellular destruction by the use of electric fields. This modality requires that electrical pulses travel from a probe to either another probe or a grounding pad that is placed in another area on the patient. The exact area of cellular destruction is not always apparent. Post treatment drug infusion performed is done by placing a needle into an area near the treatment zone. In some embodiments, the needle is not part of the system and requires user skill to place it accurately. This method may be inaccurate for drug delivery into the area of cellular lysis and may result in less effective immunogenic results.

Other methods cryoablate by placing the cryoprobe into the target tissue and causing cellular destruction by freezing the tissue with long freezing cycles lasting 8 to 10 minutes. These freezing cycles are often repeated numerous times to destroy the entirety of the tumor. A needle is then placed approximately where the ablation was performed, without accuracy and without assured co-location with the cryoprobe. It is usual that the needle is not part of the system and requires user skill to place it accurately. This method may be inaccurate and may not provide the expected immunogenic response. Further, long and repeated freeze/thaw cycles are used in cryoablation where the entirety of the tumor is destroyed. Lymphatic pathways would be destroyed as well.

In contrast to the methods noted in the prior several (5) paragraphs, the system of the present disclosure comprises a design that includes an energy probe combined and integral with an infusion sheath for infusing agents accurately into selected target. The energy probe and the agents infused, thus, are co-located in the a selected target. In some embodiments, the configuration of the infusion sheath is not dependent on the drugs to be infused. For example, the drugs infused may be of any single drug or multiple mixture of drugs as the user deems to be appropriate for the subject at that time. In other words, the infusion sheath may be agnostic to the drug or mixture of drugs passed through the sheath. In some embodiments, the infusion sheath is configured to permit a specific type of drug to pass through the sheath. For example, in a drug formulation having a higher viscosity, the infusion sheath may be formulated with a larger diameter thereby reducing the pressure needed to infuse the drug formulation into a user.

### Probe

Numerous cryosurgical instruments including cryoprobes, cryosurgical probes, cryosurgical ablation devices, cryostats and cryocoolers have been used for cryosurgery. These devices typically use the principle of Joule-Thomson expansion to generate cooling, taking advantage of the fact that cryogenic fluids, when rapidly expanded, become extremely cold. In some embodiments, a high-pressure gas mixture is expanded through a nozzle inside a small cylindrical shaft or tube typically made of steel. The Joule-Thomson expansion cools the steel tube to a cold temperature very rapidly. In some embodiments, the cooled cryosurgical probes are brought into contact with diseased tissue, forming ice balls and freezing the diseased tissue. Properly performed cryosurgical procedures allow cryoablation of the diseased tissue without undue destruction of surrounding healthy tissue.

Cryosurgical probes are used to treat a variety of diseases. Cryosurgical probes quickly freeze diseased body tissue, causing the tissue to die after which it will be absorbed by the body, expelled by the body, sloughed off or replaced by scar tissue. Cryosurgical treatment can be used to treat both benign and cancerous tissues, e.g., prostate cancer and benign prostate disease. Cryosurgery also has gynecological applications in treatment of the uterus or fibroids. In addition, cryosurgery may be used for the treatment of a number of other diseases and conditions including, but certainly not limited to, breast cancer, liver cancer, renal cancer, glaucoma and other eye diseases.

In some embodiments, a cryolysis therapy is mediated by an intratumoral cryolysis device comprising a cryoprobe type of probe for contacting and freezing tumor cells during freeze-thaw cycles. In some embodiments, a cryogen circulates within a cryoprobe. In some embodiments, a cryogen is selected from the group consisting of: argon, nitrous oxide, carbon dioxide, and liquid nitrogen. In some embodiments, administration of a cryolysis therapy comprises at least one, at least two, or at least three cycles of freeze-thaw. In some embodiments, administration of a cryolysis therapy comprises one cycle of freeze-thaw. In some embodiments, administration of a cryolysis therapy comprises two cycles of freeze-thaw. In some embodiments, administration of a cryolysis therapy comprises three cycles of freeze-thaw.

In some embodiments, the probe is an energy probe. In some embodiments, the energy source used to create cellular lysis is selected from a group consisting of ultrasound, cryolysis, radiation, radiofrequency, high intensity focused ultrasound (HIFU), microwave, pulsed electric fields, and any combination thereof. Other energy sources known to those of skill in the art may also be used to emit energy at a target site within a patient. In some embodiments, the energy probe is a cryoprobe source configured to generate cryolysis therapy by the flow of a cryogenic fluid through the probe that mediates tumor cellular lysis by cryolysis.

In some embodiments, the cryoprobe is configured to be any diameter that is commonly used in the field for intratumoral cryotherapy. In some embodiments, a cryoprobe has a diameter of about 1.5 mm to about 3.4 mm. In some embodiments, a cryoprobe has a diameter of about 1.5 mm. In some embodiments, a cryoprobe has a diameter of about 3.4 mm. In some embodiments, a cryoprobe diameter is selected to enable a rapid cooling rate. In some embodiments, achieving a rapid cooling rate requires that a cooling probe achieves a temperature of about −40° C. or colder, about −50° C. or colder, or about −60° C. or colder, in less than a minute. In some embodiments, a cryoprobe surface may reach −130° C. to −150° C. and as low as −190° C. as needed for rapid cooling of target tissue. In some embodiments, cryoprobe diameter is selected to enable efficient cryolysis of a target tissue such that the cryoprobe effectively elicits a partial necrotic zone within a target tissue, which can be a tumor, to release sufficient amounts of TSA and DAMPs to initiate an immune response. In some embodiments, the probe is coaxial with the infusion sheath. The probe sizes may range from 1 mm in outer diameter to 3 mm in outer diameter.

In a non-limiting example, the energy being delivered through the probe may create cellular lysis by necrosis, thereby disrupting the cellular walls and facilitating the release of antigens and other cellular components into the tumor micro-environment. In some embodiments, the drug infusion sheath facilitates the accurate infusion of drugs into the same location of the target site where the cellular lysis was created. In some embodiments, the probe passes through the conduit for drug infusion such that the probe and the drug infusion sheath coincide with each other. Thus, the location the drug infusion coincides with the cellular lysis treatment area and errors in directing the drug infusion into the target treatment zone are reduced or eliminated.

### Infusion Sheath

In some embodiments, the energy probe is surrounded by the an infusion sheath, and thus coaxial therewith for infusion of a fluid (i.e. the drug) into the target site, which can include target tissue or be at the treated area in instances where the probe is activated prior to the fluid infusion to the same co-located site. In some embodiments, the infusion sheath comprises a lumen coaxial with the probe which resides in such lumen. In use, in some embodiments, the probe translates distally out of the sheath into a target site (e.g. a tumor), where cryo energy or other energy from the probe is delivered to the tissue of the target site. The infusion sheath can translate distally over the probe and deliver a treatment fluid out of openings (e.g. slits or holes or elongated holes) in the distal end or in a distal portion of the infusion sheath to the target site while keeping the probe substantially stationary to co-locate the fluid with the location of energy (e.g. cryo) delivery by the probe. The delivery of the treatment fluid may occur while the infusion sheath is moving distally and/or proximally relative to the probe, or at a point or at several intermediate points when the infusion sheath is stationary relative to the probe, at the discretion of the user to co-locate the fluid relative to the probe's energy delivery.

In some embodiments, the outer diameter of the probe may be smaller than the inner diameter of the sheath, creating a space for the fluid to travel distally to exit one or more openings or ports at the distal end of the infusion sheath, which may include one or more slits or one or more holes or one or more elongated holes at a distal portion of the infusion sheath. The one or more openings or ports may be oriented to allow the fluid to exit the infusion sheath radially relative to the longitudinal axis of the probe. The one or more openings or ports may be oriented to allow the fluid to exit the infusion sheath in a direction substantially parallel to the longitudinal axis of the probe, for example, wherein the openings are at the distal tip of the infusion sheath. In some embodiments, after cellular lysis, fluid is infused through the lumen of the infusion sheath to co-locate the drug infused at the target site without moving the energy probe, maintaining accuracy of the infusion location.

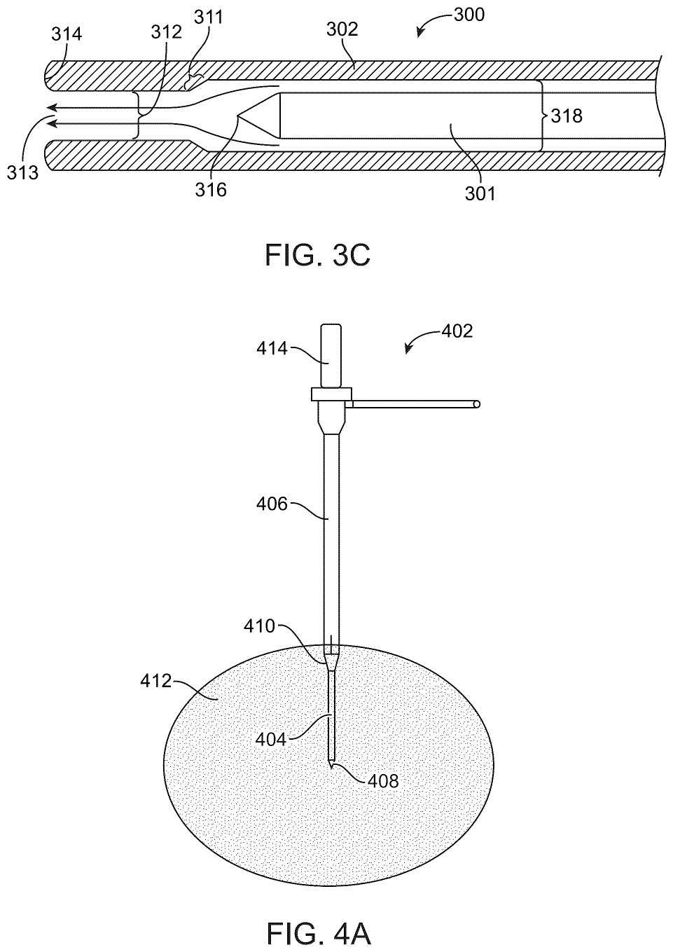

In some embodiments, the outer diameter of the probe is tapered to increase from the distal tip in a proximal direction. For example, the distal tip may have a first outer diameter that is smaller outer diameter located proximal thereto. In such embodiments, the infusion sheath may have a constant inner diameter at the distal end thereof such that the distal translation of the infusion sheath over the probe over the probe over the distal tip creates a open pathway to allow the fluid to exit the opening of the system between the infusion sheath and the probe. In some embodiments, a distal portion of the probe is a constant outer diameter, and the infusion sheath comprises an distal portion having a smaller inner diameter than a second distal portion proximal thereto, for example as described and shown in FIGS. 3A-3C.

In some embodiments, the difference in the outer diameter of the probe and the inner diameter of the sheath creates sufficient opening for the fluid to travel to the distal portion of the sheath. In some embodiments, the fluid travels distally to exit the distal tip of the sheath. In some embodiments, the one or more openings may be included in the sheath that are or extend proximal to the distal tip of the sheath. In some embodiments, the sheath is substantially tapered at the distal end such that the inner diameter of the sheath is substantially the same as the outer diameter of the probe (although still translatable relative thereto) to force fluid to exit the openings rather than the distal tip of the sheath. In some embodiments, the fluid travels distally to exit one or more openings or ports at the distal end of the infusion sheath, which may include one or more slits or one or more holes or one or more elongated holes at a distal portion of the infusion sheath. In some embodiments, the fluid travels distally to exit the distal end of the sheath. The one or more openings or ports may be oriented to allow the fluid to exit the infusion sheath radially relative to the longitudinal axis of the probe. The one or more openings or ports may be oriented to allow the fluid to exit the infusion sheath in a direction substantially parallel to the longitudinal axis of the probe, for example, wherein the openings are at the distal tip of the infusion sheath. In some embodiments, after cellular lysis, fluid is infused through the lumen of the infusion sheath to co-locate the drug infused at the target site without moving the energy probe, maintaining accuracy of the infusion location.

In some embodiments, the infusion sheath comprises two coaxial tubes forming a fluid column therebetween for the fluid to be delivered from the proximal end to a distal portion of the coaxial tubes. The infusion sheath is also coaxial with the longitudinal axis of the probe at the distal portion of the infusion sheath, at least. In some embodiments, the fluid travels distally to exit one or more openings or ports at the distal end of the infusion sheath, which may include one or more slits or one or more holes or one or more elongated holes at a distal portion of the infusion sheath. In some embodiments, the fluid travels distally to exit the distal end of the sheath. The one or more openings or ports may be oriented to allow the fluid to exit the infusion sheath radially relative to the longitudinal axis of the probe. The one or more openings or ports may be oriented to allow the fluid to exit the infusion sheath in a direction substantially parallel to the longitudinal axis of the probe, for example, wherein the openings are at the distal tip of the infusion sheath. In some embodiments, after cellular lysis, fluid is infused through the lumen of the infusion sheath to co-locate the drug infused at the target site without moving the energy probe, maintaining accuracy of the infusion location.

In some embodiments, the infusion sheath comprises a first lumen that is coaxial with the longitudinal axis of the probe, and a second lumen having a second longitudinal axis extending substantially parallel to the longitudinal axis of the probe and/or the first lumen for infusion of fluid (i.e. the drug) into the target site. As previously noted, the target site can include target tissue or be at the treated area in instances where the probe is activated prior to the fluid infusion to the same co-located site. In some embodiments, after cellular lysis, fluid is infused through the second lumen of the infusion sheath to co-locate the drug infused at the target site without moving the energy probe, maintaining accuracy of the infusion location. The infusion sheath may have one or more openings or ports at its distal end in fluid communication with the second lumen through which the fluid exits the infusion sheath. The openings or ports may be slits, holes, elongated holes or other suitable shapes, and may be positioned to infuse the fluid substantially parallel or coaxially relative to the second longitudinal axis, normally to the second longitudinal axis, radially relative to the second longitudinal axis, or in any other combination or direction.

In some embodiments, the infusion sheath is a multi-lumen tube for infusion of fluid (i.e. the drug) into the target site, which can include target tissue or be at the treated area in instances where the probe is activated prior to the fluid infusion to the same co-located site. The multiple lumens may be parallel to a central lumen through which the probe extends. In some embodiments, after cellular lysis, fluid is infused through a lumen of the infusion sheath, or through the multiple lumens of the infusion sheath (in such embodiments) to co-locate the drug infused at the target site without moving the energy probe, maintaining accuracy of the infusion location.

In some embodiments, the energy probe is retractable back into the infusion sheath. In some embodiments, the energy probe extends out the distal end of the infusion sheath for placement. In some embodiments, after cellular lysis treatment is performed the probe is retracted into the infusion sheath far enough that infused fluid can move around the end of the retracted energy probe and out the end hole of the infusion sheath, maintaining accuracy of the infusion location. Relative movement of the probe and infusion sheath is possible in the embodiments herein. Thus, while retraction of the probe into a stationary infusion sheath may be mentioned, the embodiments herein may also functionally be used with the probe stationary and the sheath moving relative thereto. In order to co-locate the fluid (i.e. drug) where the energy probe delivered its energy, where the sequence of treatment is energy delivery then drug delivery, it is the latter relative movement noted (i.e. probe stationary with sheath advancing distally over the probe to deliver fluid while advancing or after advancement) that may be more advantageous in some circumstances. Alternatively, however, it is envisioned that other locations of delivery of fluid or drug relative to the treatment site of energy delivery may be advantageous. In such instances, the embodiments described herein with co-axial delivery of the infusion sheath and its openings with the probe allows for optionality along with accuracy and control of the fluid delivery relative to the energy delivery location.

In some embodiments, the outer diameter of the infusion sheath may be 2 mm or larger. In some embodiments, the infusion sheath consists of a single or multichannel fluid path. In some embodiments, the infusion sheath may be a polymer such as PTFE, Nylon, PP, PE or metal such as stainless steel or nitinol. In some embodiments, the infusion sheath has a side arm with a tube for connecting to an infusion source, which may include a pump. In some embodiments, the infusion pump is any pump suited to transport fluids. In some embodiments, the infusion pump is a roller pump, syringe pump, syringe cartridge, or hand syringe. In some embodiments, the side arm tubing extends from the infusion sheath 30 cm to 245 cm. In some embodiments, the inner diameter of the side arm tubing is small and provides a low priming volume such that the volume required to prime the system is less than or equal to 0.01 mL/cm, or less than or equal to 0.005 mL/cm, or less than or equal to 0.0075 mL/cm, reducing potential waste of drug in the side arm tubing. This disclosure recognizes that the infusion of drug is not dependent on the type of drug being infused. In some embodiments, the drug is one or multiple drugs selected by the user that is best for the subject.

### Fluid Such as Therapeutic Agent

Traditional clinical approaches for cancer immunotherapy rely on the systemic administration of high doses of one or two biological immunomodulatory agents. Major advances in the treatment of cancer have been made in the last decade by the use of monoclonal antibodies (mAbs) that block immune checkpoint inhibitor molecules such as PD-1, PD-L1 and CTLA-4. However, despite the significant benefits obtained by the use of these mAbs, a substantial fraction of patients still do not respond to the therapy or become refractory to it. Additionally, the use of high systemic doses of these checkpoint inhibitors is accompanied by high frequency of serious adverse events, particularly when used in combination with other immunotherapeutic agents, which limits the number of agents and doses that can be combined to address the multifactorial nature of tumor immune resistance.

As described herein, any fluid may be delivered with the device. For example, the fluid may be a therapeutic agent. Any fluid may be a therapeutic agent. In some embodiments, the therapeutic agent is configured to treat cancer. For example, the therapeutic agent may be one or two biological immunomodulatory agents.

Traditional cancer immunotherapeutic approaches only provide benefit to a limited fraction of patients. This may be due to systemic delivery of drugs which is limited in effectiveness and is associated with toxicity and adverse events. The methods, systems, and apparati provided and described herein deliver drugs intratumorally and as a result, the amount of drug required is reduced compared to systemic delivery. Intratumoral delivery immediately following cellular lysis can carry the intracellular components, i.e., tumor specific antigens (TSA), tumor associated antigens (TAA), damage associated molecular patterns (DAMPs) and other cellular components released by the breakdown of the cellular wall, into the tumor environment and lymphatic system thereby initiating an immune response. By infusing immediately post cell lysis and accurately in the center of the treatment zone the entrainment of the released antigens and other cellular components may be optimized. Additionally, intratumoral administration allows for a significant reduction in dose, which reduces the probability of toxicity and adverse events. Provided herein are methods, systems, and apparati that allow a reduced dose of drug to be delivered accurately into the target cellular lysis zone in order to effectively remove cellular debris from the tumor microenvironment of cellular components that has flowed into the area due to the cellular lysis treatment.

### Target Site

As used herein the devices are suitable for treatment and co-location of energy delivery (such as cryo) and fluid (such as a drug) to a variety of target sites. The target sites may be target tissue such as cancer tissue or cancer or tumors comprising cancerous tissue. A “cancer” refers to a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. A “cancer” or “cancer tissue” may include a tumor at various stages. In certain embodiments, the cancer or tumor is stage 0, such that, e.g., the cancer or tumor is very early in development and has not metastasized. In some embodiments, the cancer or tumor is stage I, such that, e.g., the cancer or tumor is relatively small in size, has not spread into nearby tissue, and has not metastasized. In other embodiments, the cancer or tumor is stage II or stage III, such that, e.g., the cancer or tumor is larger than in stage 0 or stage I, and it has grown into neighboring tissues, but it has not metastasized, except potentially to the lymph nodes. In other embodiments, the cancer or tumor is stage IV, such that, e.g., the cancer or tumor has metastasized.

In some embodiments, the cancer comprises adenocarcinoma, adrenal cortical cancer, anal cancer, astrocytoma, bile duct cancer, bladder cancer, bone cancer, bone sarcoma, brain tumors, brain cancer, breast cancer, cervical cancer, chordoma, colorectal cancer, endometrial cancer, esophagus cancer, Ewing family of tumors, eye cancer, gallbladder cancer, gastrointestinal carcinoid tumors, gastrointestinal stromal tumors, gestational trophoblastic disease, glioblastoma, glioma, Hodgkin disease, Kaposi sarcoma, kidney cancer, renal cell carcinoma, laryngeal and hypopharyngeal cancer, liver cancer, hepatocellular carcinoma (HCC), non-small cell lung cancer, small cell lung cancer, lung carcinoid tumor, medulloblastoma, melanoma, meningioma, mesothelioma, metastatic pituitary carcinoma, nasal cavity and paranasal sinus cancer, nasopharyngeal cancer, neuroblastoma, non-Hodgkin lymphoma, non-melanoma skin cancer, oral cancer, ovarian cancer, oral cavity and oropharyngeal cancer, osteosarcoma, ovarian cancer, pancreatic cancer, penile cancer, pituitary tumors, prostate cancer, renal cell carcinoma, retinoblastoma, rhabdomyosarcoma, salivary gland cancer, sarcoma, small intestine cancer, stomach cancer, squamous cell carcinoma (including head and neck cancer), testicular cancer, throat cancer, thymus cancer, thyroid cancer, uterine sarcoma, vaginal cancer, Wilms tumor, and any combination thereof.

In some embodiments, a cancer is a solid tumor cancer. In some embodiments, a solid tumor cancer is selected from adenocarcinoma, astrocytoma, bladder cancer, bone sarcoma, breast cancer, cervical cancer, chordoma, colorectal cancer, endometrial cancer, esophageal cancer, glioblastoma, glioma, kidney cancer, liver cancer, medulloblastoma, melanoma, meningioma, mesothelioma, metastatic pituitary carcinoma, prostate cancer, neuroblastoma, non-melanoma skin cancer, non-small cell lung cancer, oral cancer, ovarian cancer, pancreatic cancer, renal cell carcinoma, retinoblastoma, sarcoma, small cell lung cancer, squamous cell carcinoma (including head and neck cancer), stomach cancer, testicular cancer, thyroid cancer, Wilms tumors, and any combination thereof. In some embodiments, a cancer is metastatic prostate cancer. In some embodiments, a cancer is metastatic breast cancer. In some embodiments, a cancer is non-small cell lung cancer.

### Systems, Methods, and Devices

As described here, a cryolysis system is configured to slidably transition between two or more positions. In some embodiments, a first position is configured such that an infusion sheath distal end is spaced apart from a distal tip of an energy probe in the distal direction by a distance. In some embodiments, a second position is configured such that the infusion sheath distal end is spaced apart from the distal tip of the energy probe in the distal direction by a distance smaller than the distance in the first position. In some embodiments, a third position is configured such that the infusion sheath distal end is spaced apart from the distal tip of the energy probe at a distance greater than 1 cm, completely covering the probe's distal tip, and allowing infusible drug to flow past the distal tip through an interior lumen of the infusion sheath. In some embodiments, the third position is not included. In some embodiments, the third position is optional.

As used herein, the terms first, second, and third position are used to indicate a configuration of the cryolysis system and relative positions of the infusion sheath and distal tip of the probe at different point in time and are not meant to denote any specific sequencing of these positions or configurations. As used herein, the terms first and second position are used to indicate a configuration of the cryolysis system at a point in time and are not meant to denote any specific sequencing of these positions. For example, in a non-limiting example, the cryolysis system is inserted into a tissue in the second position, an infusion sheath is then retracted to the first position to allow energy to be applied to the tissue, and then the sheath is moved to the third position to allow infusible drug to flow into the tissue. Similarly, in another non-limiting example, the cryolysis system is inserted into the tissue in the first position, energy is applied to the tissue via an energy probe, followed by the infusion sheath moving to the second position and further moving into the third position to allow drug to be infused. In another non-limiting example, the cryolysis system is inserted into the tissue in the first position where energy is applied to the tissues, then the infusion sheath is advanced distally to the second position where drug is infused into the tissue.

The systems, methods, and devices described herein are not limited to three positions. For example, the cryolysis system may have two positions, four position, or any other number of potential positions. The cryolysis system may have a single position in the case that infusible drug could be released while maintaining the position of the infusion sheath, such as by rotating the infusion sheath about the longitudinal axis, allowing infusible drug to flow through the infusion sheath, past the energy probe, and into the tissue. The cryolysis system may allow for delivery of the drug while the infusion sheath is moving relative to the probe, or while the probe is moving relative to the infusion sheath.

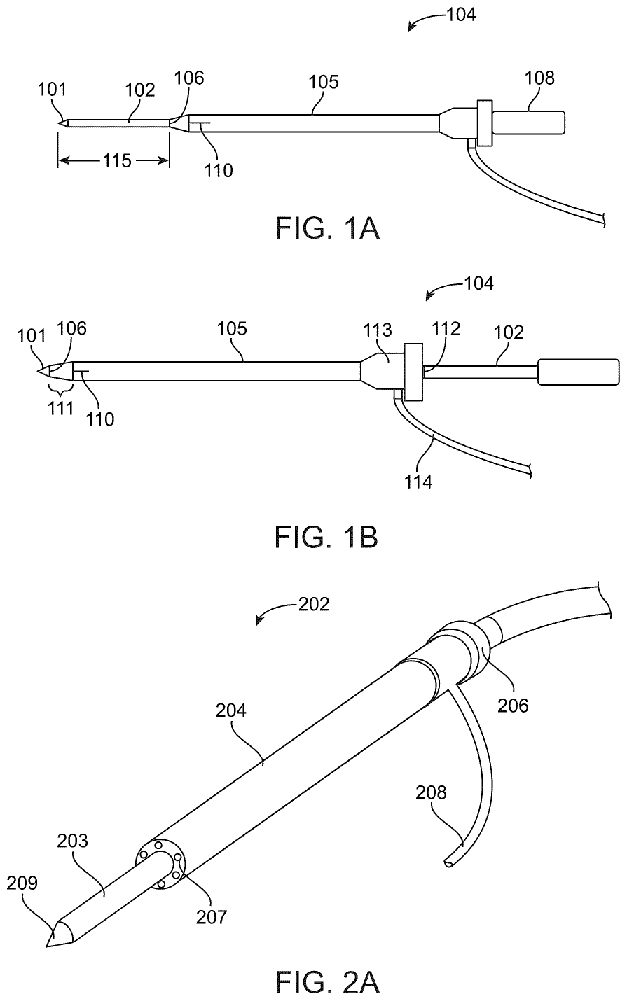

FIG. 1A illustrates an example of a cryolysis system 104 in a first position comprising probe 102 and an infusion sheath 105. When in the first position as shown in FIG. 1A, a distal tip 101 of the probe 102 extends to a first location which is distal to a second location of a sheath distal end 106. In some embodiments, the probe 102 is configured to emit energy to the surrounding tissue when in the first position. For example, the probe 102 may freeze a plurality of cells in a target tissue, thereby causing cell lysis. In some embodiments, when in the first position, the infusion sheath 105 is configured to retain an infusion of therapeutic agent. For example, the infusion sheath may be configured to fluidly couple with a reservoir containing the therapeutic agent so as to deliver the therapeutic agent from the reservoir and to the patient.

In some embodiments, the probe 102 is an energy probe. In some embodiments, the probe 102 is a cryoprobe. In some embodiments, a handle 108 is coupled with the proximal end of the probe 102. In some embodiments, the probe 102 extends to a distal tip 101. In some embodiments, the distal tip 101 is sharp. In some embodiments, the distal tip 101 is tapered up towards the distal end of the probe 102. In some embodiments, the distal tip 101 is configured to penetrate one or more different tissues in the human body. For example, the distal tip 101 may be configured to pass through skin and one or more tissues to reach the target site. In some embodiments, the distal tip 101 is round and atraumatic.

In some embodiments, the energy probe 102 extends through an infusion sheath 105. In some embodiments, the infusion sheath 105 comprises a port 110 extending through the outer surface of the infusion sheath 105 to the center lumen. In some embodiments, the port is a hole, slit, elongated hole, is circular, triangular, square, rectangular, pentagonal, heptagonal, hexagonal, or any other shape permitting flow of fluid out of the lumen of the infusion sheath 105 and to the exterior environment, which, as contemplated herein is a target site.

In some embodiments, when the infusion sheath 105 is configured to be slidably moved along the longitudinal axis of the probe 102 such that a longitudinal axis of the probe 102 and a longitudinal axis of the infusion sheath 105 are coaxial.

In some embodiments, the distal end of the infusion sheath 105 has at least one port 110 disposed through the wall of the infusion sheath 105 to communicate with the inner diameter lumen of the infusion sheath 105. For example, the at least one port 110 may be configured to permit an egress point for fluids flowing through the infusion sheath 105. In some embodiments, the at least one port 110 is configured to by disposed through the infusion sheath 105 such that loss of axial strength of the infusion sheath 105 outer wall is minimized or prevented. In some embodiments, the at least one port 110 is configured to be disposed through the infusion sheath 105 such that no material has been removed from the infusion sheath 105 outer wall when the at least one port 110 was created.

In some embodiments, the port 110 is disposed through the outer surface of the infusion sheath 105 in a longitudinal direction. In some embodiments, the port 110 is disposed through the outer surface of the infusion sheath 105 radially. In some embodiments, the at least one port 110 is disposed through the outer surface of the infusion sheath 105 radially. In some embodiments, the infusion sheath 105 comprises 1, 2, 3, 4, 5, 6, 7, 8, 9, 10, 11, 12, 13, 14, 15, 16, 18, 19, or 20 ports 110. In some embodiments, the infusion sheath 105 comprises four ports 110. In some embodiments, the infusion sheath 105 comprise two or more ports 110, where the ports 110 are distributed radial-symmetrically about the longitudinal axis of the infusion sheath 105.

In some embodiments, the distal tip 101 is spaced apart from the sheath distal end 106 in the first position by a distance 115. In some embodiments, the distance 115 is 1 cm, 2 cm, 3 cm, 4 cm, or 5 cm. In some embodiments, the distance 115 is 0 to 1 cm. In some embodiments, the distance 115 is 0 to 2 cm. In some embodiments, the distance 115 is 0 to 3 cm. In some embodiments, the distance 115 is 0 to 4 cm. In some embodiments, the distance 115 is 0 to 5 cm. In some embodiments, the distance 115 is 1 to 2 cm. In some embodiments, the distance 115 is 1 to 3 cm. In some embodiments, the distance 115 is 1 to 4 cm. In some embodiments, the distance 115 is 1 to 5 cm. In some embodiments, the distance 115 is 2 to 3 cm. In some embodiments, the distance 115 is 2 to 4 cm. In some embodiments, the distance 115 is 2 to 5 cm. In some embodiments, the distance 115 is 3 to 4 cm. In some embodiments, the distance 115 is 3 to 5 cm. In some embodiments, the distance 115 is 4 to 5 cm.

FIG. 1B illustrates an example of the cryolysis system 104 shown in FIG. 1A in a second position, according to embodiments described herein. When in the second position, the sheath distal end 106 and the distal tip 101 are spaced at a closer distance than in the first position.

In some embodiments, the cryolysis system 104 is configured to slidably move between the first position and the second position. For example, the infusion sheath 105 may be configured to slidably move in a distal direction along a longitudinal axis of the probe 102 from the first position to the second position, where the second position is spaced apart from the first position. In some embodiments, the distal tip 101 is configured to slidably move in a proximal direction from the first position to the second portion. In some embodiments, the infusion sheath 105 is configured to release an infusion of a therapeutic agent when in the second position.

In some embodiments, the cryolysis system 104 is configured to transition between a third position and the second position. In some embodiments, when in the third position, the infusion sheath 105 covers the distal tip 101 or is positioned adjacent to the distal tip 101. For example, in this third position the probe 102 coupled with the infusion sheath 105 in the third position may be configured to prevent buckling of the infusion sheath and/or provide enhanced mechanical strength relative to the energy probe alone. For example, the enhanced mechanical strength may be provided about or adjacent to the distal end of the energy probe by the close positioning of the infusion sheath 105. In some embodiments, the cryolysis system 104 is configured to slidably move from the third position to the first position, thereby exposing the distal end of the probe 102 for cell lysis.

In some embodiments, the infusion sheath 105 is slidably movable along the longitudinal axis relative to the probe 102. For example, the infusion sheath 105 may be moved in a distal direction relative to the longitudinal axis of the probe 102, thereby facilitating infusion of one or more fluids through the infusion sheath 105 and into a target site. In some embodiments, the infusion sheath 105 is configured to be moved in a distal direction relative to the longitudinal axis of the probe 102, to the target site after energy has been applied to the target site to lyse cells.

In some embodiments, the at least one port 110 is disposed in the infusion sheath 105 at a location proximal to the tapered portion 111. In some embodiments, the at least one port 110 is disposed through at least a portion of the tapered portion 111.

In some embodiments, the advancement distance of the infusion sheath 105 is determined by moving the proximal end of the sheath to align with an indicator line 112 disposed on the outer surface of the probe 102. In some embodiments, the position of the sheath distal end 106 is located such that 0 to 2.5 cm of the distal end of the probe 102 is exposed before drug infusion is started. For example, the distal end of the probe 102 may be flush with the sheath distal end 106, thereby creating a distance of 0 cm between the distal end of the probe 102 and the sheath distal end 106.

In some embodiments, the infusion sheath 105 is aligned with the indicator line 112 disposed on the outer surface of the probe 102. In some embodiments, the distal tip 101 is exposed when the cryolysis system 104 is inserted into the target tissue thereby minimizing the risk for buckling. In some embodiments, the probe 102 provides the energy necessary to create cellular lysis in the target tissue.

In some embodiments, after energy has been applied to the target tissue and cellular lysis has occurred, the infusion sheath 105 is advanced forward along the longitudinal axis of the probe 102 until the valve 113 has passed by the indicator line 112 and the indicator line 112 is visible on the probe 102. In some embodiments, the indicator line 112 is inscribed into the probe 102. In some embodiments, the indicator line 112 is a raised surface or edge that creates a snap with the inner diameter of the valve 113 or an audible click when the valve 113 passes over the indicator line 112, alerting the user that the infusion sheath 105 is in the correct position. When the infusion sheath 105 is in the correct position, the at least one port 110 is positioned with the distal tip 101 of the probe 102 in the center of the target tissue.

When the infusion sheath 105 is in the correct position, drug infusion can be performed through the tube 114. In some embodiments, fluid is pumped through the tube 114 into the infusion sheath 105. In some embodiments, the valve 113 prevents any fluid from leaking out of the proximal end of the infusion sheath 105. The fluid travels down the infusion sheath 105 until it reaches the distal end of the infusion sheath 105, where it passes through the at least one port 110 and into the target tissue. In some embodiments, the material construction of the infusion sheath 105 is made of a flexible polymer. For example, the flexible polymer may be PTFE, PP, nylon, urethane, or FEP. In some embodiments, the material construction of the infusion sheath 105 is made of a rigid polymer. For example, the rigid polymer may be HDPE or polyamide. In some embodiments, the material construction of the infusion sheath 105 is metal to allow for insertion into dense target tissues such as bone. For example, the metal may be stainless steel, nitinol, or cobalt chromium. In some embodiments, the outer diameter of the infusion sheath 105 is 1.5 mm to 2.7 mm. In some embodiments, the outer diameter of the infusion sheath 105 is 1 mm to 4 mm.

In some embodiments, the outer surface of the infusion sheath 105 has a tapered portion 111 extending between the infusion sheath 105 and the distal tip 101. For, example, a narrow end of the tapered portion 111 may interface with the distal tip 101 while a second end on the tapered portion 111 having a diameter wider than the narrow end interfaces with the infusion sheath 105 portion extending from the valve 113.

In some embodiments, the infusion sheath 105 has a valve 113 coupled to the proximal end of the infusion sheath 105. In some embodiments, the valve 113 is configured to allow fluid to flow in the infusion sheath from the proximal end of the probe 102 to the distal end of the probe 102 and prevent fluid flow in reverse. In some embodiments, the valve 113 is a hemostasis valve.

In some embodiments, a tube 114 is connected to the side of the infusion sheath 105. In some embodiments, the tube 114 is configured to allow for the introduction of fluids from an infusion pump into the infusion sheath 105. In some embodiments, the fluid being introduced can come from any type of infusion pump or syringe (not shown).

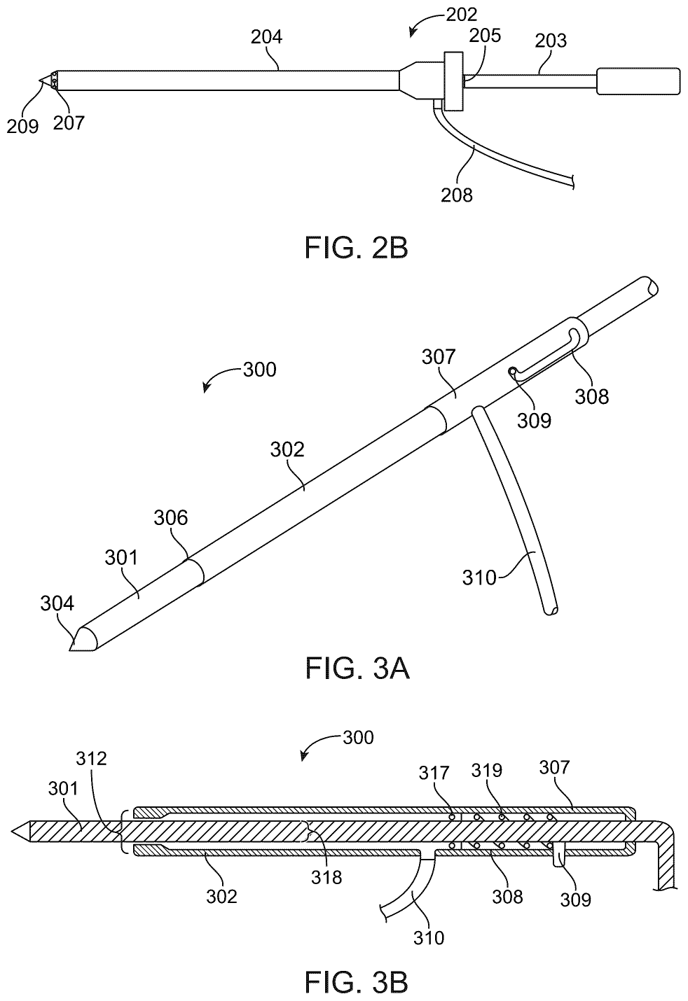

FIG. 2A illustrates an example of a cryolysis system 202 comprising a probe 203 and an infusion sheath 204 in a first position. In some embodiments, the probe 203 is an energy probe. In some embodiments, the probe 203 is a cryoprobe.

In some embodiments, the infusion sheath 204 is slidably movable along the longitudinal axis relative to the probe 203. For example, the infusion sheath 204 may be moved from the first position in a distal direction relative to the longitudinal axis of the probe 203 to a second position (see FIG. 2B), thereby facilitating infusion of one or more fluids through the infusion sheath 204 and into a target site. In some embodiments, the infusion sheath 204 is configured to be moved in a distal direction relative to the longitudinal axis of the probe 203, to the target site after energy has been applied to the target site to lyse cells. In some embodiments, the position of the sheath distal end is located such that 0 to 2.5 cm of the distal end of the probe 203 is exposed before drug infusion is started. For example, the distal end of the probe 203 may be flush with the sheath distal end, thereby creating a distance of 0 cm between the distal end of the probe 203 and the sheath distal end.

In some embodiments, the cryolysis system 202 is configured to transition between the first position and the second position by retracting the probe 203 from the first position in a proximal direction relative to the longitudinal axis of the infusion sheath 204 to reach the second position where an infusion of therapeutic agent is released. In some embodiments, the advancement distance of the infusion sheath 204 is determined by moving the proximal end of the sheath to align with an indicator line 205 disposed on the outer surface of the probe 203. In some embodiments, the cryolysis system 202 is configured to transition between a third position and the first position, as described in reference to FIG. 1A and FIG. 1B.

In some embodiments, the infusion sheath 204 has a valve 206 coupled to the proximal end of the infusion sheath 204. In some embodiments, the valve 206 is configured to allow fluid to flow in the infusion sheath 204 from the proximal end of the probe 203 to the distal end of the probe 203 and prevent fluid flow in reverse. In some embodiments, the valve 206 is a hemostasis valve. In some embodiments, end holes 207 are configured to be at the distal end of the infusion sheath 204. In some embodiments, there are a plurality of end holes 207 configured to be at the distal end of the infusion sheath 204.

In some embodiments, a tube 208 is connected to the side of the infusion sheath 204. In some embodiments, the tube 208 is configured to allow for the introduction of fluids from an infusion pump into the infusion sheath 204. In some embodiments, the fluid being introduced can come from any type of infusion pump or syringe (not shown).

In some embodiments, the probe 203 extends to a distal tip 209. In some embodiments, the distal tip 209 is sharp. In some embodiments, the distal tip 209 is round and atraumatic. In some embodiments, the distal tip 209 is tapered towards the distal end of the probe 203. In some embodiments, the distal tip 209 is configured to penetrate one or more tissue in the human body. For example, the distal tip 209 may be configured to pass through skin and one or more tissues to reach the target site. In some embodiments, the infusion sheath 204 is a multichannel tube in fluid communication with the tube 208. In some embodiments, the channels of the infusion sheath 204 run in the direction of the longitudinal axis of the infusion sheath 204. In some embodiments, the channels of the infusion sheath 204 transport fluids from the tube 208 down the longitudinal axis of the infusion sheath 204 and deliver the fluid to end holes 207.

FIG. 2B illustrates an example of the cryolysis system 202 shown in FIG. 2A in a second position. When in the second position, a sheath distal end and the distal tip 209 are spaced at a closer distance than in the first position. The position of the sheath distal end is located such that 0 to 2.5 cm of the distal end of the probe 203 is exposed before drug infusion is started. For example, the distal end of the probe 203 may be flush with the sheath distal end, thereby creating a distance of 0 cm between the distal end of the probe 203 and the sheath distal end.

In some embodiments, when the infusion sheath 204 is configured to be moved along the longitudinal axis of the probe 203 such that the infusion sheath 204 is aligned with the indicator line 205. In some embodiments, the indicator line 205 is disposed on the outer surface of the probe 203 and the distal tip 209 is exposed, the cryolysis system 202 may be inserted into the target tissue without the potential for buckling. In some embodiments, the probe 203 provides the energy necessary to create cellular lysis in the target tissue.

In some embodiments, after energy has been applied to the target tissue and cellular lysis has occurred, the infusion sheath 204 is advanced forward along the longitudinal axis of the probe 203 until the valve 206 has passed by the indicator line 205 and the indicator line 205 is visible on the probe 203. In some embodiments, the indicator line 205 is inscribed into the probe 203. In some embodiments, the indicator line 205 is a raised surface or edge that creates a snap with the inner diameter of the valve 206 or an audible click when the valve 206 passes over the indicator line 205, alerting the user that the infusion sheath 204 is in the correct position. When the infusion sheath 204 is in the correct position, the end holes 207 are positioned with the distal tip 209 of the probe 203 in the center of the target tissue.

When the infusion sheath 204 is in the correct position, drug infusion can be performed through the tube 208. In some embodiments, fluid is pumped through the tube 208 into the infusion sheath 204. In some embodiments, the valve 206 prevents any fluid from leaking out of the proximal end of the infusion sheath 204. The fluid travels down the infusion sheath 204 until it reaches the distal end of the infusion sheath 204, where it passes through the end holes 207 and into the target tissue. In some embodiments, the material construction of the infusion sheath 204 is made of a flexible polymer. For example, the flexible polymer may be PTFE, PP, nylon, urethane, or FEP. In some embodiments, the material construction of the infusion sheath 204 is made of a rigid polymer. For example, the rigid polymer may be HDPE or polyamide. In some embodiments, the material construction of the infusion sheath 204 is metal to allow for insertion into dense target tissues like bone. For example, the metal may be stainless steel, nitinol, or cobalt chromium. In some embodiments, the outer diameter of the infusion sheath 204 is 1.5 mm to 2.7 mm. In some embodiments, the outer diameter of infusion sheath 204 is 1.0 mm to 4.0 mm.

FIG. 3A illustrates another embodiment of a cryolysis system 300 comprising a probe 301 and an infusion sheath 302 in a first position. In some embodiments, when in the first position, a probe distal end 304 extends distally from a distal end of the infusible sheath 306.

In some embodiments, the cryolysis system 300 comprises a probe 301, an infusion sheath 302, and a housing 307. In some embodiments, the probe 301 is an energy probe. In some embodiments, the probe 301 is a cryoprobe. In some embodiments, the housing 307 is attached to the proximal end of the infusion sheath 302. In some embodiments, the infusion sheath 302 is configured to move slidably along the longitudinal axis of the probe 301.

In some embodiments, the housing 307 is configured with a guide slot 308 that allows the housing 307 to move about a slide 309. In some embodiments, the slide 309 is attached to the probe 301. In some embodiments, the slide 309 is used to position the housing 307 and infusion sheath 302 such that the distal end of the probe 301 is exposed or covered by the infusion sheath 302. In some embodiments, the slide 309 or the guide slot 308 is disposed in a handle of the probe 301. In some embodiments, the slide 309 is attached to the housing 307. In some embodiments, the guide slot 308 is disposed in the probe 301. In some embodiments, the slide 309 attached to the housing 307 is configured to slidably move along the guide slot 308 disposed in the probe 301.

In some embodiments, a tube 310 is configured to pass through the outer wall of the housing 307. In some embodiments, the tube 310 is configured to infuse fluid, such as an infusion of therapeutic agent into the infusion sheath 302.

FIG. 3B illustrates a cutaway profile view of the cryolysis system 300 in the first position.

In some embodiments, the cryolysis system 300 is left in the target tissue after cellular lysis has been created. In some embodiments, the probe 301 is retracted from the target tissue by sliding the slide 309 along the guide slot 308 from the distal position to a proximal position. In some embodiments, the probe 301 is slidably moved distally towards the target tissue by sliding the slide 309 along the guide slot 308 from the proximal position to the distal position.

In some embodiments, when the slide 309 reaches the proximal position on the guide slot 308, the slide 309 is moved into the side branch of the guide slot 308 and clicks into place, alerting the user that the probe 301 has been fully retracted from the target tissue and into the infusion sheath 302. In some embodiments, the distal end of the infusion sheath 302 contains a taper 311 that increases the second inner diameter 312 along the longitudinal axis of the infusion sheath 302 towards to the proximal end such that when the probe 301 is retracted there is enough space between the second inner diameter 312 of the infusion sheath 302 and the outer diameter of the probe 301 to allow for fluid flow 313 past the distal tip of the probe 301.

In some embodiments, fluid flow 313 is introduced to the infusion sheath 302 through the tube 310. In some embodiments, fluid flow 313 travels down the length of the infusion sheath 302 and exits at the distal end of the infusion sheath 302 into the target tissue after the probe 301 has been retracted.

FIG. 3C illustrates an example of a cutaway view of the distal tip of the cryolysis system 300 in a second position. As shown in FIG. 3C, the probe 301 is entirely within the infusion sheath 302 when in the second position. In some embodiments, the probe 301 is slidably moved in a proximal direction relative to the longitudinal axis of the infusion sheath 302 thereby transitioning from the first position to the second position. In some embodiments, the infusion sheath 302 is slidably moved in a distal direction relative to the longitudinal axis of the probe 301 such that the infusion sheath distal end 314 is located distal to the probe distal end 316. In some embodiments, the cryolysis system 300 is configured to transition between a third position and the first position, as described in reference to FIG. 1A and FIG. 1B.

In some embodiments, the housing 307 contains a member 317 that acts as a retaining ring and seal at the proximal end of the housing 307. In some embodiments, the member 317 is configured to prevent fluids infused through the tube 310 from leaking out the proximal end of the infusion sheath 302. Foe example, the infusion sheath 302 may have a first inner diameter 318 and a second inner diameter 312 smaller than and located distally to the first inner diameter 318.

In some embodiments, the housing 307 contains a spring 319 inside the housing 307. In some embodiments, the spring 319 is coiled around the probe 301. In some embodiments, the spring 319 maintains the position of the probe 301 relative to the housing 307 when the slide 309 is moved either forward or backwards. When the slide 309 is moved into the forward position, the distal end of the probe 301 protrudes from the infusion sheath 302 at a distance distal to the distal end of the infusible sheath 306. In some embodiments, when the slide 309 is moved into the backwards position, the distal end of the probe 301 is covered by the infusion sheath 302.

In some embodiments, the outer diameter of the probe 301 is configured to be a near line to line fit with a second inner diameter 312 of the infusion sheath 302. In some embodiments, the protrusion of the probe 301 creates a seal with the second inner diameter 312 of the infusion sheath 302. In this position, the cryolysis system 300 may be inserted into a target tissue. Once in place, the probe 301 may be activated to delivery energy and create cellular lysis in the target tissue.

In some embodiments, the infusion sheath 302 having a first inner diameter 318 located at a proximal end of the infusion sheath and second inner diameter 312 located at a distal end of the infusion sheath. In some embodiments, first inner diameter 318 extends to a taper 311 located at a location between the distal end of the infusion sheath 302 and the proximal end of the infusion sheath 302 connecting the first inner diameter 318 region with the second inner diameter 312 region of the lumen of the infusion sheath 302. In some embodiments, the first inner diameter 318 is larger than the second inner diameter 312.

In some embodiments, the second inner diameter 312 is substantially the same as the outer diameter of the probe 301. For example, when a portion of the distal end of the probe 301 is disposed within the second inner diameter 312, fluid does not flow from the inner lumen of the infusion sheath 302 through the distal end of the infusion sheath 302. In some embodiments, the first inner diameter 318 is larger than the outer diameter of the probe 301. For example, when the probe 301 is at least partially disposed only in the portion of the probe 301 having the first inner diameter 318 and not in the second inner diameter 312, fluid in the inner lumen of the infusion sheath 302 fluid flow 313 exits the distal end of the infusion sheath 302.

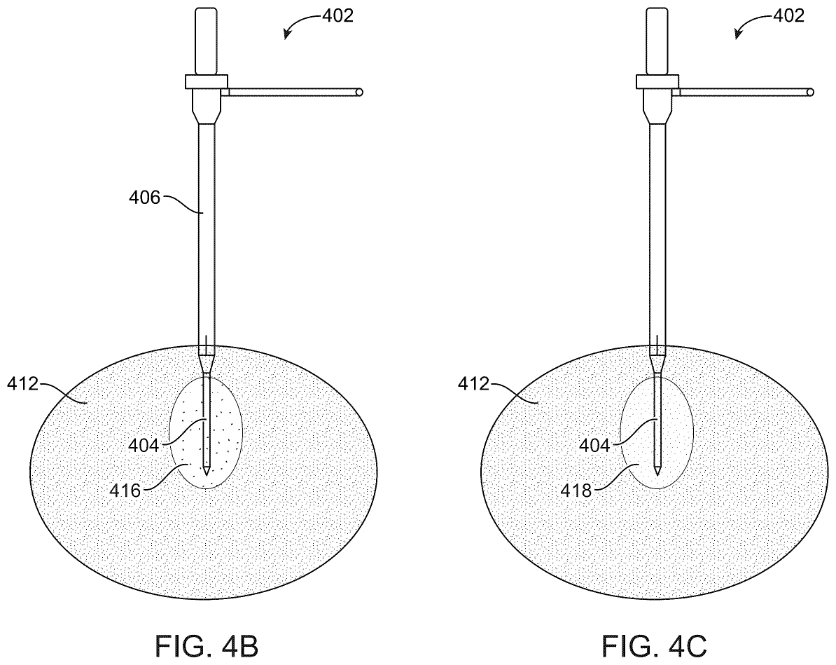

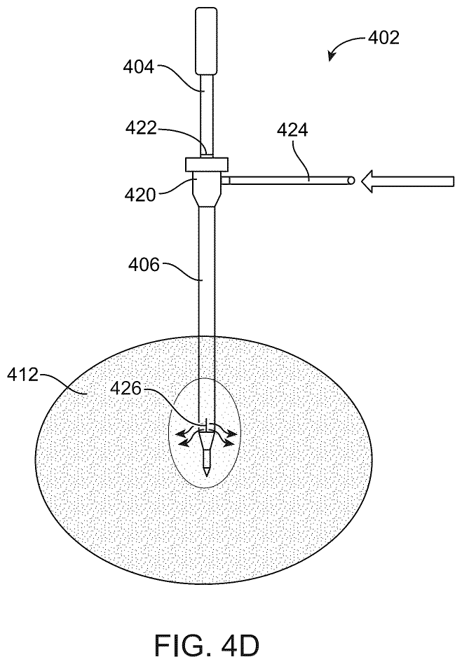

FIG. 4A-FIG. 4D illustrate a method for treating a target tissue by co-locally delivering energy and an infusion of therapeutic agent, and systems and methods thereof.

FIG. 4A illustrates an example of a cryolysis system 402 comprising a probe 404 and an infusion sheath 406 in a first position, according to embodiments described herein. When in the first position the distal tip 408 extends distally from the sheath distal end 410. In some embodiments, the distance between the distal tip 408 and the sheath distal end 410 is 1 cm, 2 cm, 3 cm, 4 cm, or 5 cm. In some embodiments, the distance between the distal tip 408 and the sheath distal end 410 is 0 cm to 1 cm. In some embodiments, the distance between the distal tip 408 and the sheath distal end 410 is 0 cm to 2 cm. In some embodiments, the distance between the distal tip 408 and the sheath distal end 410 is 0 cm to 3 cm. In some embodiments, the distance between the distal tip 408 and the sheath distal end 410 is 0 cm to 4 cm. In some embodiments, the distance between the distal tip 408 and the sheath distal end 410 is 0 cm to 5 cm. In some embodiments, the distance between the distal tip 408 and the sheath distal end 410 is 1 cm to 2 cm. In some embodiments, the distance between the distal tip 408 and the sheath distal end 410 is 1 cm to 3 cm. In some embodiments, the distance between the distal tip 408 and the sheath distal end 410 is 1 cm to 4 cm. In some embodiments, the distance between the distal tip 408 and the sheath distal end 410 is 1 cm to 5 cm. In some embodiments, the distance between the distal tip 408 and the sheath distal end 410 is 2 cm to 3 cm. In some embodiments, the distance between the distal tip 408 and the sheath distal end 410 is 2 cm to 4 cm. In some embodiments, the distance between the distal tip 408 and the sheath distal end 410 is 2 cm to 5 cm. In some embodiments, the distance between the distal tip 408 and the sheath distal end 410 is 3 cm to 4 cm. In some embodiments, the distance between the distal tip 408 and the sheath distal end 410 is 3 cm to 5 cm. In some embodiments, the distance between the distal tip 408 and the sheath distal end 410 is 4 cm to 5 cm.

In some embodiments, the method includes identifying a target tissue 412 for treatment in some embodiments, the target tissue is a cancerous tumor. In some embodiments, the method includes disposing a portion of the cryolysis system 402 into the target tissue 412 such that the distal tip 408 of the probe 404 is within the target tissue 412. In some embodiments, at least a portion of the probe 404 and at least a portion of the infusion sheath 406 are disposed in the target tissue 412. In some embodiments, the probe 404 comprises a handle 414 located at the proximal end of the probe 404. In some embodiments, the cryolysis system 402 would be inserted into the target tissue 412 in this position. In some embodiments, the probe 404 is a cryoprobe.

In some embodiments, the method includes the steps of disposing a portion of a cryolysis system 402 comprising probe 404 coupled with an infusion sheath 406 at least partially into a target tissue 412 such that a distal tip 408 of the probe 404 is disposed in the target tissue 412. In some embodiments, the cryolysis system 402 is in the first position, a second position, or a third position when disposing in the target tissue 412. In some embodiments, the cryolysis system 402 is configured to transition between a third position and the first position, as described in reference to FIG. 1A and FIG. 1B.

In some embodiments, the method includes disposing a probe 404 of a cryolysis system 402 at least partially into a target tissue 412 of a target site such that a distal tip 408 of the probe 404 is disposed in the target tissue 412. An infusion sheath 406 of the system 402 may be positioned proximal to probe and proximal to the target tissue, and then be advanced distally to the target site and infuse the fluid into the target site and, therefore, the into target tissue 412. In some embodiments the infusion sheath may substantially bathe the target tissue in the fluid such that the fluid extends beyond the edges of the target tissue (i.e. tumor).

FIG. 4B illustrates an example of a step in the method for treating a target tissue by co-locally delivering energy and an infusion of therapeutic agent with the cryolysis system 402 where at least a portion of the target tissue 412 is lysed by energy applied by the probe 404. In some embodiments, once in position, the probe 404 is ready to delivery energy to the target tissue 412 in order to impart cellular lysis. In some embodiments, cryogenic fluid is delivered to the probe 404 to create a zone of cellular lysis 416. In some embodiments, the method includes the step of providing energy from the probe 404 to the target tissue 412 thereby lysing at least a portion of a plurality of cells in the target tissue 412.

FIG. 4C illustrates an example of a step in the method for treating a target tissue by co-locally delivering energy and an infusion of therapeutic agent with the cryolysis system 402 where the area frozen by the probe 404 forming the zone of cellular lysis 416 is thawed into a zone of lysed cells 418. In some embodiments, after the zone of cellular lysis 412 has been established within the target tissue 412 and the ice ball has totally dissipated, a zone of lysed cells 418 is created.

FIG. 4D illustrates an example of a step in the method for treating a target tissue by co-locally delivering energy and an infusion of therapeutic agent with the cryolysis system 402 where the cryolysis system 402 has transitioned to a second position. In some embodiments, the method includes slidably moving the infusion sheath 406 in a distal direction along a longitudinal axis of the probe 404 from the first position to the second position, wherein the second position is spaced apart from the first position. In some embodiments, the distance between the distal tip 408 and the sheath distal end 410 is 1 cm, 2 cm, 3 cm, 4 cm, or 5 cm. In some embodiments, the distance between the distal tip 408 and the sheath distal end 410 is 0 cm to 1 cm. In some embodiments, the distance between the distal tip 408 and the sheath distal end 410 is 0 cm to 2 cm. In some embodiments, the distance between the distal tip 408 and the sheath distal end 410 is 0 cm to 3 cm. In some embodiments, the distance between the distal tip 408 and the sheath distal end 410 is 0 cm to 4 cm. In some embodiments, the distance between the distal tip 408 and the sheath distal end 410 is 0 cm to 5 cm. In some embodiments, the distance between the distal tip 408 and the sheath distal end 410 is 1 cm to 2 cm. In some embodiments, the distance between the distal tip 408 and the sheath distal end 410 is 1 cm to 3 cm. In some embodiments, the distance between the distal tip 408 and the sheath distal end 410 is 1 cm to 4 cm. In some embodiments, the distance between the distal tip 408 and the sheath distal end 410 is 1 cm to 5 cm. In some embodiments, the distance between the distal tip 408 and the sheath distal end 410 is 2 cm to 3 cm. In some embodiments, the distance between the distal tip 408 and the sheath distal end 410 is 2 cm to 4 cm. In some embodiments, the distance between the distal tip 408 and the sheath distal end 410 is 2 cm to 5 cm. In some embodiments, the distance between the distal tip 408 and the sheath distal end 410 is 3 cm to 4 cm. In some embodiments, the distance between the distal tip 408 and the sheath distal end 410 is 3 cm to 5 cm. In some embodiments, the distance between the distal tip 408 and the sheath distal end 410 is 4 cm to 5 cm. In some embodiments, the method includes the steps of delivering an infusion of a therapeutic agent to the target tissue 412 from the infusion sheath 406. In some embodiments, the method includes the steps of removing the probe 404 from the target tissue 412 prior to delivering the fluid (i.e. the therapeutic agent) to the target tissue 412.

In some embodiments, the infusion sheath 406 is advanced forward such that the proximal end of the valve 420 is in line with the indicator line 422 on the probe 404 surface. In some embodiments, the probe 404 remains in place and acts as a guide for the infusion sheath 406 to be placed in the same location as the probe 404 and the zone of lysed cells 418. In some embodiments, the side tube 424 of the infusion sheath 406 is connected to a syringe that has been filled with an infusion of a therapeutic agent i.e., one or more immunotherapeutic drug. In some embodiments, the total volume of infusion of the therapeutic agent is 15 mL. In some embodiments, the total volume of infusion of the therapeutic agent is between 5 mL to −10 mL, 10 mL to 15 mL, 15 mL to 20 mL, or 20 mL to 25 mL. In some embodiments, the total volume of infusion of the therapeutic agent is between 1 mL to 10 mL, 10 mL to 20 mL, or 20 mL to 30 mL. In some embodiments, the syringe containing the drug is installed into a syringe pump and the system is primed with drug by advancing the syringe pump until drug fills the side tube 424.

In some embodiments, the infusion syringe pump is set to a flow rate of 3 mL/min. The pump is then started with the entire volume of 15 ml being infused in 5 minutes. In some embodiments, the valve 420 at the proximal end of the infusion sheath 406 prevents infused drug from leaking out of the proximal end of the infusion sheath 406. In some embodiments, the infusion is directed to flow out of one or more slits 426 in the distal end of the infusion sheath 406. In some embodiments, the valve 420 is configured to keep the infusion sheath 406 in position, not allowing it to move along the longitudinal axis of the probe 404.

In some embodiments, when the infusion pump has completed its infusion cycle and delivered the 15 mL out of the syringe, the syringe is disconnected and the side tube 424 is flushed with saline to infuse the remaining drug in the side tube 424 tube into the zone of lysed cells 418. In some embodiments, the volume of therapeutic agent is sufficient to both prime the infusion pump and deliver remaining volume of therapeutic agent to the zone of lysed cells 418 without flushing the side tube 424 with saline to infuse the remaining drug.

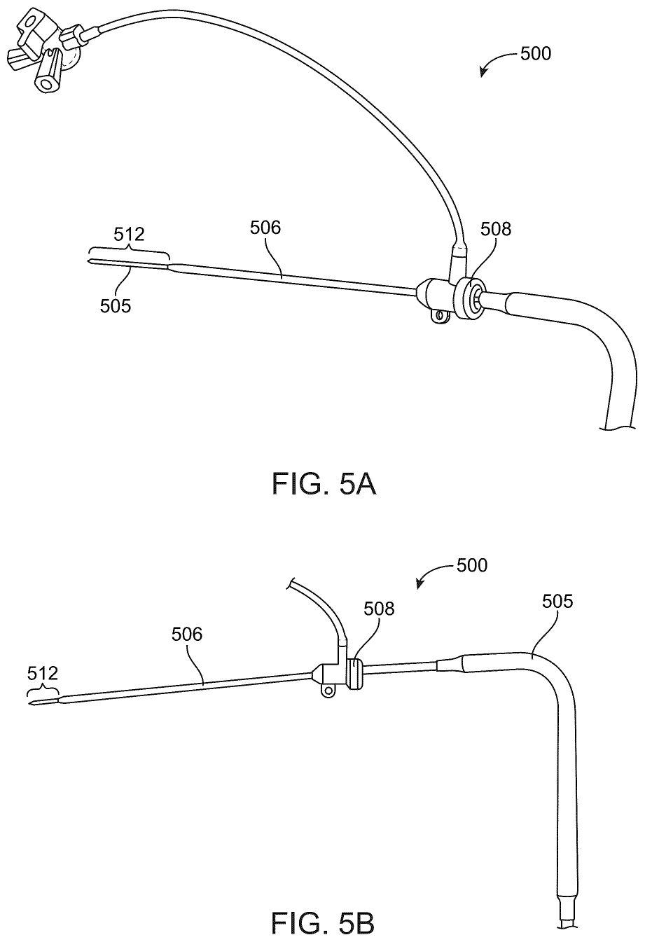

FIG. 5A illustrates an example of a cryolysis system 500 comprising a cryoprobe 505 and an infusion sheath 506 in a first position, according to embodiments described herein. In some embodiments, the cryolysis system 500 made from off the shelf components. In some embodiments, the cryolysis system 500 comprises an infusion sheath 506, a cryoprobe 505, and a valve 508. In some embodiments, the valve 508 is a hemostasis valve. In some embodiments, there is a cryoprobe tip 512 at the distal end of the cryoprobe 505 that can be inserted into a target tissue. In some embodiments, the infusion sheath 506 and the cryoprobe tip 512 are configured to have a snug fit at the location where the two meet, preventing any tissue from getting caught between the infusion sheath 506 and cryoprobe 505 when the cryolysis system 500 is inserted into the target tissue.

FIG. 5B illustrates a second view of the cryolysis system 500 shown in FIG. 5A in a second position, according to embodiments described herein. In some embodiments, the infusion sheath 506 is slidably moved in a distal direction along the longitudinal axis of the cryoprobe 505 towards the target tissue such that distance between the distal end of the infusion sheath 506 and the cryoprobe 505 is smaller than in the first position. In some embodiments, the cryoprobe 505 is slidably moved in a proximal direction relative to the longitudinal axis of the cryoprobe 505 away from the target tissue to move the cryolysis system 500 from the first position to the second position. In some embodiments, the cryoprobe 505 is completely retracted. For example, the cryoprobe 505 may be retracted after cryolysis a portion of the target tissue and before an infusion of therapeutic agent is delivered through the 506.

In some embodiments, when the cryoprobe 505 is retracted from the infusion sheath 506 fluid can be infused into the target tissue after cryolysis has been performed and the infusion sheath 506 has been advanced to the second position of the cryoprobe 505. In some embodiments, the infusion sheath 506 has holes or slits at the distal end to allow fluid to exit the cryolysis system 500 into the target tissue. In some embodiments, the valve 508 prevents fluid from leaking out the proximal end of the infusion sheath 506.

In some embodiments, the infusion sheath 506 comprises an elongate, flexible tubular body, having a proximal end, a distal end and a central lumen. In some embodiments, the valve 508 is provided in a housing on the proximal end of the infusion sheath 506. In some embodiments, the valve 508 comprises a collapsible tubular sidewall defining a valve lumen in communication with the central lumen of the infusion sheath 506.

In some embodiments, the inside diameter of the tubing maybe be continuously controlled from the collapsible valve lumen's original fully opened inside diameter to fully closed, sufficiently to clinically eliminate leakage of fluid flow through the valve 508 with or without a dilator, guide wire(s), and/or a catheter in the valve lumen. This enables the valve 508 to function with no leaking of air or liquid at any state between fully closed and fully open as needed.

In some embodiments, the valve 508 is shipped with a retention feature such as a pin or clip to keep the valve 508 open between production and use. In some embodiments, it is desirable to hold the valve 508 open for one or more steps such as to reduce friction with the cryoprobe 505 as it is being advanced or retracted through the valve 508. A retention feature or clip or control on the housing, which may be in the form of a handle, may be provided to permit selective, temporary locking of the valve 508 open during the procedure as may be desired.

### Selected Embodiments

Embodiment 1: A system for collocating an energy for cell lysis and an infusion of a fluid in a target site including tissue of a subject, the system comprising:
a. an energy probe configured to emit an energy into the tissue of the target site thereby causing cell lysis of said tissue; and
b. an infusion sheath having a lumen configured to at least partially surround the energy probe, wherein the infusion sheath is configured to provide a pathway for the fluid to travel distally within the lumen and exit a distal portion of the infusion sheath,

wherein the probe is coaxial with the infusion sheath and shares a longitudinal axis thereby allowing co-location of the energy from the probe and the infusion of the fluid from the infusion sheath in the target site.

Embodiment 2: The system according to embodiment 1, wherein the infusion sheath comprises at least one port in the distal portion of the infusion sheath that is in fluid communication with the pathway of the infusion sheath.

Embodiment 3: The system according to embodiment 1 or 2, wherein the at least one port is a slit.

Embodiment 4: The system according to any one of embodiments 1 to 3, wherein the energy probe and the infusion sheath are translatable relative to one another along the longitudinal axis.

Embodiment 5: The system according to any one of embodiments 1 to 4, wherein the pathway and port are configured to deliver the fluid while the probe is within the pathway at the distal end of the infusion sheath.

Embodiment 6: The system according to any one of embodiments 1 to 5, comprising a valve configured to eliminate proximal flow of the fluid during and/or following delivery thereof to the target site.

Embodiment 7: The system according to any one of embodiments 1 to 6, comprising a tube in fluid communication with the pathway of the infusion sheath at a distal end of the tube and in fluid communication with a source of the fluid at a proximal end of the tube.

Embodiment 8: The system according to any one of embodiments 1 to 7, wherein the fluid is a therapeutic agent effective in treating the tissue at the tissue site.

Embodiment 9: The system according to embodiment 8, wherein the therapeutic agent is a cancer therapeutic agent

Embodiment 10: The system according to any one of embodiments 1 to 9, wherein the energy induces cryolysis of tissue at the target site.

Embodiment 11: The system according to any one of embodiments 1 to 10, comprising a location indicator configured to provide a visual, tactile, or audible indication of the probe's position relative to the infusion sheath's position or vice versa.

Embodiment 12: The system according to any one of embodiments 1 to 11, comprising a location indicator configured to provide a visual, tactile, or audible indication of a position of a port of the infusion sheath relative to the probe's position, or vice versa.

Embodiment 13: The system according to any one of embodiments 1 to 12, comprising a channel disposed in the exterior surface of the energy probe, the channel configured to slidably couple with a protrusion extending from the interior wall of the infusion sheath, thereby preventing circumferential rotation of the infusion sheath relative to the energy probe.

Embodiment 14: The system according to any one of embodiments 1 to 13, comprising a channel disposed at least partially through a wall of the infusion sheath, the channel configured to slidably couple with a protrusion extending from the exterior surface of the energy probe, thereby preventing circumferential rotation of the infusion sheath relative to the energy probe.

Embodiment 15: The system according to any one of embodiments 1 to 14, comprising a valve configured to connect an infusion tube with a proximal end of the infusion sheath, so as to provide the infusion of the fluid to the enter lumen of the infusion sheath and/or a plurality of fluid channels disposed within the walls of the sheath.

Embodiment 16: The system according to any one of embodiments 1 to 15, wherein the infusion sheath has a first inner diameter located at a proximal end of the infusion sheath and a second inner diameter located at a distal end of the infusion sheath.

Embodiment 17: The system according to embodiment 16, wherein the first inner diameter is larger than the second inner diameter, and wherein the first inner diameter tapers to the second inner diameter in a location between a proximal end of the infusion sheath and the distal end of the infusion sheath.

Embodiment 18: The system according to embodiment 17, wherein the second inner diameter forms a fluid tight seal with the outer diameter of the energy probe, such that when a portion of the energy probe is in a first position disposed within the first region of the infusion sheath having the second inner diameter, the fluid does not flow from the inner lumen of the infusion sheath through the distal end of the infusion sheath, and wherein, when the energy probe is in a second position at least partially disposed the second region of the infusion sheath having the first inner diameter and not in the first region of the infusion sheath the infusion sheath having the second inner diameter, the fluid in the inner lumen of the infusion sheath is in fluid communication with the target site.

Embodiment 19: The system according to any one of embodiments 1 to 18, comprising an indicator disposed at least partially on or about the energy probe.

Embodiment 20: The system according to embodiment 19, wherein the indicator is a visual distance and/or rotational indicator located on the system that indicates to a user that a distal end of the infusion sheath has been advanced to a location relative to the probe that co-locates the fluid delivery with the energy delivered by the probe.

Embodiment 21: The system according to any one of embodiments 1 to 20, wherein the indicator is an audio indicator configured to notify a user that the distal end of the infusion sheath has been advanced to a location relative to the probe that co-locates the fluid delivery with the energy delivered by the probe.

Embodiment 22: The system according to any one of embodiments 1 to 21, wherein the indicator marks a target location on a proximal end of the system to slidably position the infusion sheath relative to the energy probe to deliver the fluid in a location that is co-located with the energy delivery location of the probe.

Embodiment 23: The system according to any one of embodiments 1 to 22, wherein the infusion sheath is slidable relative to the longitudinal axis of the energy probe.

Embodiment 24: The system according to any one of embodiments 1 to 23, wherein the energy probe is slidable relative to the longitudinal axis of the infusion sheath.

Embodiment 25: The system according to any one of embodiments 1 to 24, wherein the energy probe is a cryoprobe.

Embodiment 26: The system according to any one of embodiments 1 to 25, wherein the infusion sheath comprises a plurality of fluid pathways disposed within a wall of the infusion sheath, wherein the fluid pathways are configured to release the infusion of the therapeutic agent to flow from a first location of the infusion sheath through a second location of the infusion sheath spaced apart from and located distal to the first location.

Embodiment 27: The system according to any one of embodiments 1 to 26, wherein the second location is flush with the distal end of the infusion sheath.

Embodiment 28: The system according to any one of embodiments 1 to 27, wherein the at least one port is an annular orifice.

Embodiment 29: The system according to any one of embodiments 1 to 28, wherein the infusion sheath comprises a polymer.

Embodiment 30: The system according to any one of embodiments 1 to 29, wherein the infusion sheath comprises a metal.

Embodiment 31: The system according to any one of embodiments 1 to 30, wherein the distance the infusion sheath is slidably moved over the energy probe or the distance the energy probe is moved while at least partially within the infusion sheath is controlled by moving a guide within a slot to a target distance, wherein the guide extends from the energy probe or the infusion sheath and the slot is at least partially disposed in or extending from the corresponding surface of the energy probe or the infusion sheath.

Embodiment 32: The system according to any one of embodiments 1 to 31, wherein the infusion sheath has a first inner diameter located at a proximal end of the infusion sheath and a second inner diameter located at a distal end of the infusion sheath.

Embodiment 33: The system according to embodiment 32, wherein the first inner diameter is larger than the second inner diameter, and wherein the first inner diameter tapers to the second inner diameter in a location between the proximal end of the infusion sheath and the distal end of the infusion sheath.

Embodiment 34: The system according to embodiment 33, wherein the second inner diameter is substantially the same as the outer diameter of the energy probe, such that when a portion of the energy probe is disposed within the second inner diameter, fluid does not flow from the inner lumen of the infusion sheath through the distal end of the infusion sheath, and wherein the first inner diameter is larger than the outer diameter of the energy probe, such that when the energy probe is at least partially disposed only in the portion of the energy probe having the first inner diameter, the inner lumen of the infusion sheath is in fluid communication with the target site.

Embodiment 35: The system according to any one of embodiments 1 to 34, wherein the infusion sheath comprises a plurality of fluid pathways disposed through a wall of the infusion sheath, wherein the fluid pathways are in fluid communication with a source of the fluid at the proximal end of the system, and each pathway is in fluid communication with an associated opening at the distal end of the infusion sheath.

Embodiment 36: The system according to embodiment 35, wherein the opening is selected from a group consisting of a slit and an annular orifice.

Embodiment 37: A method for co-locating energy delivery and fluid delivery to tissue at a target site in a patient, the method comprising: disposing a cryolysis system comprising an energy probe coaxial with and positioned within a lumen of an infusion sheath at least partially into a patient such that a distal end of the energy probe is disposed in the target site; providing energy from the energy probe to the tissue thereby lysing at least a portion of a plurality of cells in the target site; slidably moving the infusion sheath in a distal direction along a longitudinal axis of the energy probe from a first position to a second position, wherein the second position is spaced apart from the first position; delivering an infusion of a fluid to the target site through the infusion sheath; and removing the infusion sheath and the energy probe from the target site.

Embodiment 38: The method of embodiment 37, comprising delivering the fluid out one or more openings in the distal end of the infusion sheath, wherein such openings are disposed radially through a wall of the infusion sheath to be in fluid communication with a fluid pathway within a lumen of the infusion sheath that extends to a proximal end of the cryolysis system to a fluid source.

Embodiment 39: The method of embodiment 38, wherein the one or more openings is a slit.

Embodiment 40: The method of any one of embodiments 37 to 39, wherein delivering the fluid and slidably moving the infusion sheath are done concurrently.

Embodiment 41: The method of embodiment 40, comprising slidably moving the infusion sheath in a proximal direction along the longitudinal axis of the energy probe from the second toward the first position, wherein delivering the fluid and slidably moving in this proximal direction are done concurrently.

Embodiment 42: The method of embodiment 41, comprising slidably moving the infusion sheath distal to the second position to a third position, wherein when in such third position buckling of the infusion sheath is reduced relative to when in the first or second positions and/or mechanical strength of the system at the location of the energy probe is enhanced relative to the energy probe alone without the sheath distal advancement, wherein the enhanced mechanical strength is provided about the distal end of the energy probe upon disposing the cryolysis system in the patient.

Embodiment 43: The method of any one of embodiments 37 to 42, wherein the infusion sheath is in the third position when disposed at least partially into the patient.

Embodiment 44: The method of any one of embodiments 37 to 43, wherein a distal end of the infusion sheath is located distal to a distal end of the energy probe when in the third position.

Embodiment 45: The method of any one of embodiments 37 to 44, wherein a distal end of the infusion sheath is located proximal to a distal end of the energy probe when in the third position.

Embodiment 46: The method of any one of embodiments 37 to 45, wherein a distal end of the infusion sheath is located at a location distal to the distal end of the energy probe when in the second position.

Embodiment 47: The method of any one of embodiments 37 to 46, wherein a distal end of the infusion sheath is located at a location proximal to the distal end of the energy probe when in the second position.

Embodiment 48: The method of any one of embodiments 37 to 47, comprising sliding a protrusion extending from the exterior wall of the energy probe within a channel disposed in the infusion sheath to slidably move the infusion sheath from the first position to the second position, wherein the channel is configured to slidably couple with the protrusion, and optionally, wherein the protrusion coupled with the channel is configured to prevent circumferential rotation of the infusion sheath relative to the energy probe.

Embodiment 49: The method of any one of embodiments 37 to 48, comprising sliding a protrusion extending from the exterior surface of the energy probe within a channel disposed at least partially through a wall of the infusion sheath to slidably move the infusion sheath from the first position to the second position, wherein the channel is configured to slidably couple with the protrusion, and optionally, wherein the protrusion coupled with the channel is configured to prevent circumferential rotation of the infusion sheath relative to the energy probe.

Embodiment 50: The method of any one of embodiments 37 to 49, comprising determining the second position by aligning a portion of the proximal end of the sheath relative to a portion of a proximal end of probe according to an audio, tactile, or visual indicator.

Embodiment 51: The method of embodiment 50, wherein the indicator marks a target location to slidably align the infusion sheath and the energy probe thereby co-locating delivery of the fluid with the energy delivered by the probe.

Embodiment 52: The method of any one of embodiments 37 to 51, wherein delivering the infusion of a therapeutic agent to the target tissue occurs through a valve configured to connect an infusion tube with a proximal end of the infusion sheath, so as to provide fluid through a pathway within the infusion sheath and out the one or more openings of the infusion sheath.

Embodiment 53: The method of any one of embodiments 37 to 52, wherein the energy probe is a cryoprobe and providing energy to the tissue at the tissue site at least partially freezes the tissue thereby lysing at least a portion of the plurality of cells in the target site.

Embodiment 54: The method of any one of embodiments 37 to 53, wherein delivering the infusion of a fluid to the target tissue comprises opening a valve at the proximal end of the system in the fluid pathway of the fluid that connects an infusion fluid source with a proximal end of the infusion sheath.

Embodiment 55: The method of any one of embodiments 37 to 54, wherein the infusion sheath is configured to form a seal with the energy probe when in the first position such that the infusion of therapeutic agent is retained in the infusion sheath, and wherein the infusion sheath is configured to deliver the infusion of therapeutic agent to the target tissue when in the second position.

Embodiment 56: The method of any one of embodiments 37 to 55, wherein a distal end of the infusion sheath is located proximal to the distal end of the energy probe when in the first position.

Certain examples of the present disclosure were described above. It is, however, expressly noted that the present disclosure is not limited to those examples, but rather the intention is that additions and modifications to what was expressly described herein are also included within the scope of the disclosed examples. Moreover, it is to be understood that the features of the various examples described herein were not mutually exclusive and may exist in various combinations and permutations, even if such combinations or permutations were not made express herein, without departing from the spirit and scope of the disclosed examples. In fact, variations, modifications, and other implementations of what was described herein will occur to those of ordinary skill in the art without departing from the spirit and the scope of the disclosed examples. As such, the disclosed examples are not to be defined only by the preceding illustrative description.

In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of examples has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner and may generally include any set of one or more limitations as variously disclosed or otherwise demonstrated herein.

What is claimed is:

1. A system for collocating an energy for cell lysis and an infusion of a fluid in a target site including tissue of a subject, the system comprising:
(a) an energy probe configured to emit an energy into the tissue of the target site thereby causing cell lysis of said tissue; and
(b) an infusion sheath having a lumen configured to at least partially surround the energy probe, wherein the infusion sheath is configured to provide a pathway for the fluid to travel distally within the lumen and exit a distal portion of the infusion sheath,
wherein the probe is coaxial with the infusion sheath and shares a longitudinal axis thereby allowing co-location of the energy from the probe and the infusion of the fluid from the infusion sheath in the target site.

2. The system according to claim 1, wherein the infusion sheath comprises at least one port in the distal portion of the infusion sheath that is in fluid communication with the pathway of the infusion sheath.

3. The system according to claim 2, wherein the at least one port is a slit.

4. The system according to claim 1, wherein the energy probe and the infusion sheath are translatable relative to one another along the longitudinal axis.

5. The system according to claim 1, wherein the path way and port are configured to deliver the fluid while the probe is within the pathway at the distal end of the infusion sheath.

6. The system according to claim 1, comprising a valve configured to eliminate proximal flow of the fluid during and/or following delivery thereof to the target site.

7. The system according to claim 1, comprising a tube in fluid communication with the pathway of the infusion sheath at a distal end of the tube and in fluid communication with a source of the fluid at a proximal end of the tube.

8. The system according to claim 1, wherein the fluid is a therapeutic agent effective in treating the tissue at the tissue site.

9. The system according to claim 8, wherein the therapeutic agent is a cancer therapeutic agent

10. The system according to claim 1, wherein the energy induces cryolysis of tissue at the target site.

11. A method for co-locating energy delivery and fluid delivery to tissue at a target site in a patient, the method comprising:
disposing a cryolysis system comprising an energy probe coaxial with and positioned within a lumen of an infusion sheath at least partially into a patient such that a distal end of the energy probe is disposed in the target site;
providing energy from the energy probe to the tissue thereby lysing at least a portion of a plurality of cells in the target site;
slidably moving the infusion sheath in a distal direction along a longitudinal axis of the energy probe from a first position to a second position, wherein the second position is spaced apart from the first position;
delivering an infusion of a fluid to the target site through the infusion sheath; and
removing the infusion sheath and the energy probe from the target site.

12. The method of claim 11, comprising delivering the fluid out one or more openings in the distal end of the infusion sheath, wherein such openings are disposed radially through a wall of the infusion sheath to be in fluid communication with a fluid pathway within a lumen of the infusion sheath that extends to a proximal end of the cryolysis system to a fluid source.

13. The method of claim 12, wherein the one or more openings is a slit.

14. The method of claim 11, wherein delivering the fluid and slidably moving the infusion sheath are done concurrently.

15. The method of claim 14, comprising slidably moving the infusion sheath in a proximal direction along the longitudinal axis of the energy probe from the second toward the first position, wherein delivering the fluid and slidably moving in this proximal direction are done concurrently.

16. The method of claim 15, comprising slidably moving the infusion sheath distal to the second position to a third position, wherein when in such third position buckling of the infusion sheath is reduced relative to when in the first or second positions and/or mechanical strength of the system at the location of the energy probe is enhanced relative to the energy probe alone without the sheath distal advancement, wherein the enhanced mechanical strength is provided about the distal end of the energy probe upon disposing the cryolysis system in the patient.

17. The method of claim 16, wherein the infusion sheath is in the third position when disposed at least partially into the patient.

18. The method of claim 16, wherein a distal end of the infusion sheath is located distal to a distal end of the energy probe when in the third position.

19. The method of claim 16, wherein a distal end of the infusion sheath is located proximal to a distal end of the energy probe when in the third position.

20. The method of claim 11, wherein a distal end of the infusion sheath is located at a location distal to the distal end of the energy probe when in the second position.
